SEQUENCE LISTING (11 sequences)

SEQ ID NO: 1 - DNA, 240 bp, Homo sapiens
tgacctaataatgatgggttttatttccagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaa

SEQ ID NO: 2 - protein, 64 aa, Homo sapiens
TSLLMVIMGELEPSEGKIKHSGRISFCSQFSWIMPGTIKENIIFGVSYDEYRYRSVIKACQLEE

SEQ ID NO: 3 - DNA, 16 bp, Homo sapiens
tctcagttttcctgga

SEQ ID NO: 4 - DNA, 18 bp, Homo sapiens
gggtaaaattaagcacag

SEQ ID NO: 5 - DNA, 17 bp, Homo sapiens
atagatacagaagcgtc

SEQ ID NO: 6 - DNA, 18 bp, Homo sapiens
ctagaagaggtaagaaac

SEQ ID NO: 7 - DNA, 41 bp, Homo sapiens
accgcatcgaatgcatgtctcgggatctcagttttcctgga

SEQ ID NO: 8 - DNA, 18 bp, Homo sapiens
gtttcttacctcttctag

SEQ ID NO: 9 - DNA, 42 bp, Homo sapiens
cgattccgctccagacttctcgggagacgcttctgtatctat

SEQ ID NO: 10 - DNA, 33 bp, Homo sapiens
cccgaattcggcaccattaaagaaaatatcatt

SEQ ID NO: 11 - DNA, 35 bp, Homo sapiens
cccgaattcggcaccattaaagaaaatatcatcct

(19) United States
(12) Patent Application Publication
(10) Pub. No.: US 2004/0072208 A1
(43) Pub. Date: Apr. 15, 2004

(54) Surface acoustic wave sensors and method for detecting target analytes

(76) Inventors: Peter Warthoe, Copenhagen (DK); Soerensen Iben, Copenhagen N (DK)

Correspondence Address: Dorsey & Whitney LLP, Intellectual Property Department, Suite 3400, Four Embarcadero Center, San Francisco, CA, 94111-4187, US

(21) Appl. No.: 10/441,643

(22) Filed: May 20, 2003

Related U.S. Application Data
(60) Provisional application No. 60/383,247, filed on May 23, 2002.

(51) Int. Cl. C12Q 1/68

(52) U.S. Cl. 435/6; 205/777.5

(57) ABSTRACT

The present invention relates generally to methods and compositions for analyzing binding molecules including proteins and nucleic acid molecules. In addition the invention relates to the use of surface acoustic wave sensors that rely on non-fluorescent detection system consisting of a sensor using propagation of surface acoustic waves for detection of a wide variety of biological-based assays.

### FIELD OF THE INVENTION

The present invention relates generally to methods, compositions and devices for analyzing molecules including proteins and nucleic acid molecules. The invention relates to the use of surface acoustic wave s for the detection of molecules.

### BACKGROUND OF THE INVENTION

Detection and analysis of biological molecules including nucleic acid molecules are among the most important techniques in biology. A number of methods have been developed which permit the implementation of extremely sensitive assays based on nucleic acid detection. Most of these methods employ amplification of targets or probes. These include the polymerase chain reaction (PCR), ligase chain reaction (LCR), self-sustained sequence replication (3SR), nucleic acid sequence based amplification (NASBA), strand displacement amplification (SDA), and amplification with Q-beta-replicase (Birkenmeyer and Mushahwar, J. Virological Methods, 35:117-126 (1991); Landegren, Trends Genetics, 9:199-202 (1993)) and Rolling Circle Amplification, RCA (Landegren U, Nucleic-Acids Res. 1998 Nov 15:26(22):5073-8), all of which are expressly incorporated herein by reference.

If the analysis of nucleic acid molecules is to continue being useful in practical diagnostic applications it is desirable to assay for many targets simultaneously. Such multiplex assays are typically used to detect five or more targets. It is also desirable to obtain accurate quantitative data for the targets in these assays. In a multiplex assay, it is especially desirable that quantitative measurements of different targets accurately reflect the true ratio of the target sequences.

Generally, following essentially all biochemical reactions, analysis entails some form of detection step. Of special interest is the detection of nucleic acid hybridizations and antibody-antigen binding reactions. Ideally, detection should be sensitive. It should allow processing of multiple samples and should not include any form for modification of the biological material. In addition, it should be quite easy and fast to use at routine basis. The last two requirements are particularly important if the technology should be widespread including locations where advanced molecular biology equipment are not available e.g. a medical doctor practice or bio-clinical laboratory for routine molecular diagnostics blood testing. However, current detection techniques are somewhat limited in these characteristics.

Hybridization of nucleic acid molecules is generally detected by autoradiography or phosphor image analysis when the hybridization probe contains a radioactive label or by densitometer when the hybridization probe contains a label, such as biotin or digoxin, that is recognized by an enzyme-coupled antibody or ligand.

When a radiolabeled probe is used, detection by autoradiography suffers from film limitations, such as reciprocity failure and non-linearity. These film limitations can be overcome by detecting the label by phosphor image analysis. However, radiolabels have safety requirements, increasing resource utilization and necessitating specialized equipment and personnel training. For such reasons, the use of nonradioactive labels has been increasing in popularity. In such systems, nucleotides contain a label, such as biotin or digoxin, which can be detected by an antibody or other molecule that is labeled with an enzyme reactive with a chromogenic substrate. Alternatively, fluorescent labels may be used. These systems do not have the safety concerns as described above, but use components that are often labile and may yield nonspecific reactions, resulting in high background (i.e., low signal-to-noise ratio). One major disadvantage of the above described labeling methods is the need for modification of the biological material. This makes them not very attractive outside high specialized genetics laboratories.

Antibody-antigen binding reactions may be detected by one of several procedures. As for nucleic acid hybridization, a label, radioactive or nonradioactive, is typically conjugated to the antibody. The types of labels are similar: enzyme reacting with a chromogenic substrate, fluorescent, hapten that is detected by a ligand or another antibody, and the like. As in detection of nucleic acid hybridization, similar limitations are inherent in these detection methods. In general all detection methods known today require a modification of the molecule e.g DNA or RNA or protein that should be detected. This makes the current detection methods very labor intensive and in general not very user friendly since many steps are required before the final result are obtained.

The polymerase chain reaction (PCR) is a method for specific amplification of DNA fragments. The simplicity and high efficiency of the reaction makes it not only a very powerful research method, but also a very reliable and sensitive diagnostic tool for detection of nucleic acids of different pathogen or nucleic acid sequence information such as various genotypes or single nucleotide polymorphisms. The PCR has been utilized many times in the diagnosis of numerous diseases. However, this reaction, although efficient and simple has not found a substantial niche in the diagnostic laboratories around the world. The basic PCR techniques are described in U.S. Pat. No. 4,683,195 and 4,683,202 to Mullis, et al., the disclosures of which are incorporated herein. While these techniques have found widespread use in biology, their usefulness in clinical applications has been principally limited by three factors, to wit: (1) conventional PCR does not yield quantitative data it because the amount of nucleic acid increases exponentially and plateaus; (2) it will occasionally amplify nonspecific nucleic acids, and (3) the PCR products must be assessed by semi-quantitative methods such as Southern blotting and densitometry. As a result, most PCR assays are limited to use in applications where the presence or absence of a specific, known nucleic acid molecule (usually DNA) is to be determined.

Researchers have developed various methods intended to allow for quantification of PCR-amplified DNA or RNA. Generally, these approaches involve amplification followed by size analysis on agarose gels or DNA/RNA hybridizations followed by isotopic or enzymatic detection. For example, in Proc. Ntl. Acad. Sci. USA, (1992) 89:3241-3245, a method was reported involving heat (rather than alkaline) denaturation of the PCR product and hybridization in solution of the separated strands to two oligonucleotide probes. One probe is biotin labeled (a “capture” probe); the other is labeled with horseradish peroxidase (HRP) (a “detector” probe). Solution hybridization of the PCR product strands to the probes is performed in microtiter plate wells. These plate wells are coated with streptavidin hydrophobically bound thereto which is intended to bind with the biotinylated probe. After washing, an HRP chromogen is added to the wells, absorbance is measured by a microtiter plate reader and ratios of PCR product separately bound by the probes are measured against a standard curve. One major reason of this delayed acceptance of the PCR in practical diagnostics is inefficient methods for the detection of the PCR products. A common way of detection is agarose gel electrophoresis. This method requires relatively large amounts of the amplified DNA. To obtain this large amount of DNA the PCR is usually carried out through many cycles of amplification, which makes the reaction very sensitive to cross-contamination of treated specimens, or increases non-specific products.

These non-specific products can lead to misinterpretation of the results. In addition, gel electrophoresis detection of PCR products is not amenable to the needs of routine diagnostic laboratories, which are unlikely to have appropriate equipment. In addition the method is time consuming and suffers from the inability to perform high-throughput screening.

Moreover, PCR results are generally interpreted by visual analysis of a band stained with ethidium bromide, which is a subjective method requiring highly qualified staff. As a result, many attempts to design a calorimetric nonisotopic method for the detection of PCR products analogous to immunological reactions for enzyme immunoassay (EIA) have been attempted. Colorimetric reactions are much more sensitive, can be measured by simple photometers, and can be quantitative allowing more reliable and more objective interpretation of the results.

A notable difficulty with colorimetric approaches for detection is the unavailability of a specific method to capture the PCR products. There are three different currently available ways to capture PCR products: (1) hybridization with a probe attached to a solid-phase (microtiter well), (2) antibodies specific to RNA-DNA hybrids, which can be prepared to specifically capture hybrids formed between amplified DNA and specific RNA probes, and (3) specific labeling of the PCR products (usually biotinylation) by using special labeled primers, or nucleotides. Only hybridization with a probe provides sequence specific capture of the PCR fragments. However, the main disadvantage of hybridization is low efficiency of the process because of high dependence on DNA denaturation conditions. At annealing temperatures or at neutralization conditions after alkali denaturation, DNA forms a double-stranded structure. If the double-stranded DNA is denatured it can hybridize with an oligonucleotide probe and the product can be captured and detected; however, if the DNA is not denatured it cannot be captured. Thus, the usual hybridization techniques are inefficient, since three different competing reactions occur simultaneously when standard annealing conditions are used: (1) probe binding, (2) restoration of the double-stranded form of the PCR fragments, and (3) nonspecific burial of the interacting region of the amplified DNA product inside of the macrostructure organized in the DNA.

To overcome two of the major challenges in PCR detection: a) the quantitative data challenges and b) new detection method, there has recently been developed a new method for real time detection of the PCR product. The new fluorescent assay system are based on the 5′ exonuclease activity of Taq DNA polymerase has been developed for detecting correctly amplified targets produced during the polymerase chain reaction (PCR). The method uses an oligonucleotide probe complementary to an internal region of the target sequence and included into each PCR reaction. The probe contains a fluorescent dye and a quencher. During the extension phase of PCR, Taq polymerase releases the dye from the quencher, thus increasing fluorescent yield of the dye. The assay is at least as sensitive as ethidium bromide staining, and eliminates the need for analysis of PCR products by gel electrophoresis. Completed PCR reactions are read in a luminescence spectrometer equipped with a microwell plate reader. Data is collected automatically and transferred to a spreadsheet.

Recently the rolling circle amplification technology has become an alternative to PCR for applications involving the detection of specific nucleic acid sequences. The method involves amplifying a circular nucleic acid probe produced following interaction of a nucleic acid probe with a target sequence whereby the circular nucleic acid probe is enriched prior to amplification. Enrichment reduces the level of background amplification by removing any linear nucleic acid probes, and may be enzymatic or non-enzymatic. Amplification may be by rolling circle amplification. The probe may be a padlock probe. The terminal sequences of the probe may form non-contiguous duplexes with the probe circularized through ligation of a capture ligand or spacer nucleic acid molecule between the two terminal sequences. The capture ligand or spacer nucleic acid molecule may be labeled, such as with biotin. RCA assays are described in more detail in BMC Genomics (2001) 2:4, which is expressly incorporated herein by reference.

In summary both relatively well characterized methods such as PCR and more newly developed methods such as RCA all still require modification of the biological material before detection. Generally detection requires relatively expensive highly specialized equipment not available in a typical physician's office or bio-clinical laboratory for routine molecular diagnostics blood testing.

The present invention provides novel compositions and methods which are utilized in a wide variety of nucleic acid-based procedures, and further provides other, related advantages.

### SUMMARY OF THE INVENTION

In accordance with the objects outlined above, the present invention provides a method for determining the presence or absence of a target nucleic acid in a test sample comprising contacting a target nucleic acid comprising first and second adjacent regions with a surface acoustic wave sensor comprising a surface having an immobilized probe nucleic acid which hybridizes to said first region of the test nucleic acid to form a hybridization complex, wherein the first region of the target nucleic acid is double stranded and the adjacent second region of the target nucleic acid is single stranded in the hybridization complex. The method further includes extending the probe nucleic acid in the hybridization complex using the second region in the test nucleic acid as template, applying an input signal to an input transducer to generate a surface acoustic wave within the surface acoustic wave sensor, receiving the surface acoustic wave at an output transducer, generating an electronic output signal, and measuring a parameter of said output signal which provides an indication of whether or not the target nucleic acid is present in the test sample.

The invention includes sensors to determine the presence or absence of a target analyte comprising a microsensor wherein the microsensor has a surface, at least a portion of which is capable of binding to a target analyte. A surface acoustic wave is generated in the surface, and propagates across a binding area. Upon binding of the analyte, the propagation of the surface acoustic wave is altered, and the alteration is detected electronically.

In one embodiment a set of interdigitated input electrodes is in communication with a piezoelectric layer. The application of an electronic input signal to the input electrodes generates a surface acoustic wave in the piezoelectric layer. A set of interdigitated output electrodes in communication with the piezoelectric layer detects the surface acoustic wave in the form of an output signal. Binding events are detected by comparing the input signal to the output signal. A change in frequency between the input and output signal—a frequency shift—is indicative of a binding event. In other embodiments, a binding event may be detected as a phase shift or amplitude change.

In other embodiments, a reference sensor is provided. In the reference sensor, a surface acoustic wave is generated by an input transducer and traverses a surface where no binding events occur. The reference surface acoustic wave is detected in the form of a reference output signal by an output transducer. A binding event is detected by comparing the output surface acoustic wave from the active sensor to the output surface acoustic wave from the reference sensor.

In other embodiments, output signals from one or more active sensors are compared to indicate a binding event.

In some embodiments, differential circuitry is provided for comparing the output signal from an active sensor to that of a reference sensor, or for comparing the output signals from two or more active sensors.

In another aspect, the invention further includes an oscillator and an oscillator controller for generating a signal that, when applied to the input electrodes generates a surface acoustic wave.

In general, at least two surface acoustic wave sensors—one active and one reference—are used. An active SAW sensor is treated with an agent which specifically binds to a target analyte and is the measuring sensor whereas another sensor is not so treated and is referred to as a reference sensor. The reference sensor is used as need be to correct for non-specific environmental factors such as mass flow, temperature and the like.

In other embodiments, the reference sensor is treated with a binding agent, but the sample solution applied to the reference sensor does not contain any target analyte suitable for binding the agent bound to the reference sensor.

In still other embodiments, an array of active sensors is provided along with at least one reference sensor.

In some embodiments, the reference sensor may be integrated with one or more active sensors. That is, a reference and active sensor may comprise the same piezoelectric and substrate layer. In other embodiments, a reference sensor and active sensor may be separate devices and operatively associated through electronic circuitry.

One or more of the aforementioned sensors can be incorporated into a microfluidic device. In such embodiments, at least one sensor is positioned in a microfluidic channel or chamber wherein fluid flows past the surface of the microsensor. A multiplicity of such microsensors each having different analyte specificity can be incorporated into the channel and/or chamber for multiplex analyte analysis of a test sample.

Another aspect the invention is directed to a method for determining the presence or absence of a target analyte, such as a nucleic acid or protein, in a test sample. In the case of nucleic acids, in one embodiment, the method comprises contacting the target nucleic acid with an active sensor comprising a microsensor having a surface which comprises an immobilized probe nucleic acid which hybridizes to a first region of the test nucleic acid. When so bound, a hybridization complex is formed. The formation of this complex and therefore the presence of the target analyte can be detected by comparing an input signal to an output signal, or comparing an output signal of an active sensor to an output signal from a reference sensor. In general, a reduction in frequency is indicative of a binding event. However, in other embodiments, a phase or amplitude shift may be utilized to detect binding.

In the hybridization complex formed by the immobilized probe and test nucleic acid, the first region of the target nucleic acid is hybridized to the immobilized probe and forms a double-stranded region. A second region of the target nucleic acid, adjacent to the first region, is single-stranded in the hybridization complex. The hybridization complex is then exposed to a condition (e.g., nucleotide extension via a polymerase or oligonucleotide ligation via a ligase) which results in the extension of the probe nucleic acid in the hybridization complex using the second region in the test nucleic acid region as template. Thereafter a parameter of the piezoelectric element or a laser is used to provide an indication of whether or not the probe nucleic acid has been extended.

In a further aspect, the probe nucleic acid comprises a terminal end region comprising the last 3 nucleotides and preferably a terminal nucleotide which in one embodiment contains one or more base pair matches or mismatches with the opposing nucleotide(s) in the first region of the test nucleic acid in the hybridization complex. In another embodiment, the base pair matching or mismatching occurs in the second region of the test nucleic acid. For example, in the case of oligonucleotide hybridization to the second region and subsequent ligation (IEOLA), base pair matches or mismatches may be in the end region of the probe or the end region of the oligonucleotide adjacent to the immobilized probe. In either case, it is preferred that the match or mismatch occur at the terminal nucleotide portion. Extension of the immobilized primer provides an indication of the sequence present in the target nucleic acid complementary to said end regions.

In conjunction with detection of nucleic acids, optionally, an amplification reaction such as PCR or LCR may be performed prior to or simultaneously with the contacting of the target nucleic acid with the biosensor.

Other embodiments which provide sequence information include a polymerase based probe extension wherein the separate addition of one or more of the possible nucleotide triphosphates results in selective probe extension.

In addition, just as primer extension can be detected on a SAW sensor, sequencing can be performed on a SAW sensor. In this embodiment, each nucleotide that is added to the primer is detected on the SAW sensor. As such, the sequence of the target can be obtained.

In addition, proteins can be detected either directly or indirectly. When directly detected a protein affinity agent is immobilized on the biosensor. The sensor is then contacted with a sample that potentially contains the target protein. Upon binding of the target to the affinity agent, a change in output signal is detected as an indication of the presence of the target.

When indirectly detected, the protein is generally contacted in solution with an affinity agent that is coupled to a nucleic acid. Affinity agents, as described herein include but are not limited to aptamers, antibodies and ligands. Affinity agents are coupled to a detection moiety. Generally the detection moiety is a nucleic acid. Following removal of unbound affinity agents, the detection moiety nucleic acid is amplified forming amplicons. Amplicons are then detected on the biosensor as an indication of the presence of the protein.

### BRIEF DESCRIPTION OF THE DRAWINGS

FIG. 1 depicts the surface acoustic wave sensor design.

FIG. 1a depicts a schematic drawing of a SAW sensor having an interdigitated input transducer (a) and one interdigitated output transducer (b). A DNA extension reaction is on-going on the surface between the two IDT sets (c).

FIG. 1b a photograph of a commercial available SAW filter having an interdigitated input transducer (a) and one interdigitated output transducer (b). The DNA or protein hybridisation arel take place on the surface between the two IDT sets (c).

FIG. 2 depicts the protein measuring principle.

FIG. 2a depicts that the anti-human IL-6 antibody is covalently assembled on the sensing surface between two interdigital transducers.

FIG. 2b a photograph of a commercial available SAW filter used for covalent assembled the IL-6 antibody.

FIG. 2c depicts hybridization with the test protein complex consisting of IL-6 anti-human antibody/IL-6 molecules/IL-6 biotinylated anti-human IL-6 antibody/avidin-horseradish peroxidase conjugate.

FIG. 2d depicts that the protein complex is formed on the sensor surface and the acoustic wave will be delayed relative to the receiver interdigital transducers (IDTs) with a reduction of frequency as the end result.

FIG. 3 depicts the sensitivity of a SAWS-IL-6-Biosensor. Frequency shift after injection of 100 μl sample mix containing 1.5 pg, 4.5 pg, 5.5 pg, 7.5 pg and 10 pg of recombinant IL-6.

FIG. 4 depicts the sensitivity of a SAWS-SNP-Biosensor. Time dependent frequency change in the presence of a wt probe and a wt target DNA. A 100 μl DNA mixture having 1 pg of a 599 bp fragment was injected into the SAWS-SNP-Biosensor. The first part of the curve (a) corresponds to the hybridization even between the probe/target DNA molecules. The second part of the curve (b) corresponds to the time-dependent frequency change upon building of double stranded DNA molecules on the sensor surface, the DNA extension reaction.

FIG. 5 depicts the selectivity of the SAWS-SNP-Biosensor. Measurement of time dependent frequency changes after injection of (a) DNA from blood with wt CFTR gene, (b) DNA from blood with heterozygous CFTR gene, (c) DNA from blood with homozygous CFTR gene. 5×3 disposables SAWS-SNP-Biosensors were used, one for each blood sample. The SAWS-SNP-Biosensor was programmed with a wt probe. After testing five blood samples within each category, a characteristic curve was identified in each category (wt, heterozygous, homozygous). Approx. 20 min. after injection each curve was stabilized at a given frequency. The characteristic frequency shift for the three sample types are shown in FIG. 5.

FIG. 6 depicts the nucleic acid measuring principle.

FIG. 6a A DNA probe is attach to the sensing surface between the two interdigital transducers.

FIG. 6b Single stranded DNA is hybridised to the specific probe on the sensing surface

FIG. 6c The double stranded DNA molecule is build on the sensing surface if a perfect match between the 3′ end of the probe and the single stranded DNA exist.

FIG. 6d The double stranded DNA is not build on the surface since a mismatch exist on the 3′ end position of the attached DNA probe.

FIG. 7 Strand Displacement Amplification, The target generation step

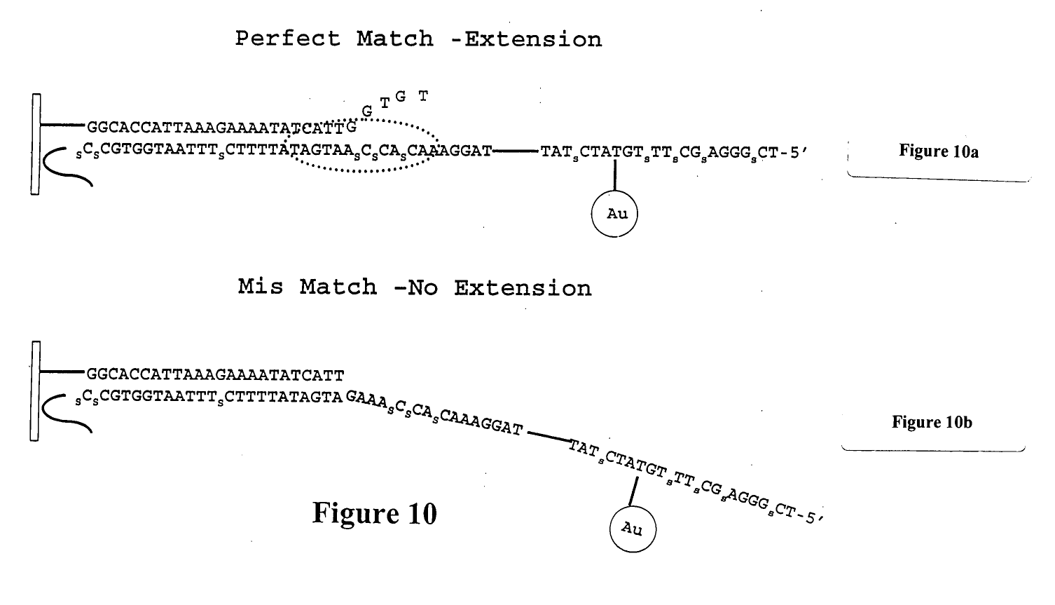

FIG. 8 Strand Displacement Amplification, The amplification step

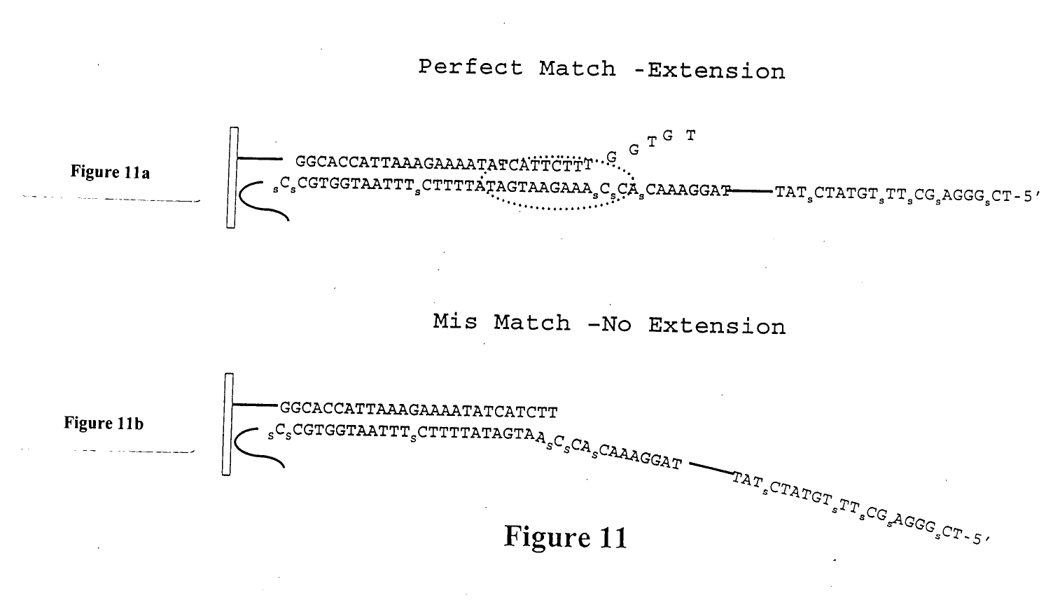

FIG. 9a,b DNA extension on SAW sensing surface using Strand

### DISPLACEMENT AMPLIFICATION

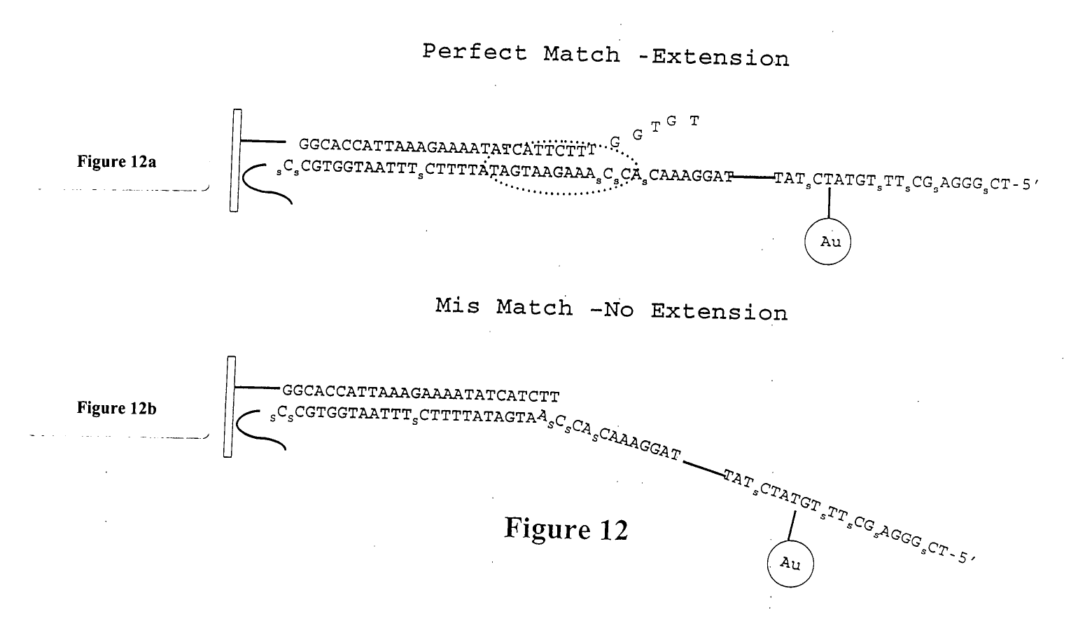

The CFTR detector probe 1 is immobilized to the Surface Acoustic Wave (SAW) biosensor surface. The target sequence is amplified by SDA in the solution. The complementary amplified target sequences, generated with the S2 Amplification primer, hybridize to the immobilized CFTR detector probe 1 at the SAW surface (FIG. 9a). If a perfect match is present (D508), Bst DNA polymerase will extend the 3′ end of the immobilized CFTR detector probe 1 and a double-stranded DNA will be generated at the SAW surface. If a mismatch (wt gene) at the 3′-end is present, no extension will occur and the single-stranded DNA, generated with S2 amplification primer will remain single-stranded (FIG. 9b).

FIG. 10 Gold particle are linked to the Thymidine with an Amine group. The target sequence is amplified by SDA, using the S2 Amplification primer labeled with gold particle and the amplification primer S1 (table 1) and Bumper primer B1 and B2 (table 1). The complementary amplified target sequence, generated with the S2 Amplification primer linked with a gold particle, hybridize to the immobilized CFTR detector probe 1 at the SAW surface. If a perfect match (D508) is present, Bst DNA polymerase will extend the 3′ end of the immobilized CFTR detector probe 1 and a double-stranded DNA will be generated at the SAW surface (FIG. 10a). If a mismatch (normal gene) at the 3′-end is present, no extension will occur and the single-stranded DNA, generated with S2 amplification primer will remain single-stranded (FIG. 10b).

FIG. 11 The CFTR detector probe 2 is immobilized to the Surface Acoustic Wave (SAW) biosensor surface. The target sequence is amplified by SDA in the solution. The complementary amplified target sequences, generated with the S2 Amplification primer, hybridize to the immobilized CFTR detector probe 2 at the SAW surface. If a perfect match (normal gene) is present, Bst DNA polymerase will extend the 3′ end of the immobilized CFTR detector Probe 2 and a double-stranded DNA will be generated at the SAW surface (FIG. 11a). If a mismatch (D508) at the 3′-end is present, no extension will occur and the single-stranded DNA, generated with S2 amplification primer will remain single-stranded (FIG. 11b).

FIG. 12 Gold particle are linked to the Thymidine with an Amine group. The target sequence is amplified by SDA, using the S2 Amplification primer labeled with gold particle and the amplification primer S1 (table 1) and Bumper primer B1 and B2 (table 1). The complementary amplified target sequence, generated with the S2 Amplification primer linked with a gold particle, hybridize to the immobilized CFTR detector probe 2 at the SAW surface (FIG. 12). If a perfect match (normal gene) is present, Bst DNA polymerase will extend the 3′ end of the immobilized CFTR detector probe 1 and a double-stranded DNA will be generated at the SAW surface (FIG. 12a). If a mismatch (D508) at the 3′-end is present, no extension will occur and the single-stranded DNA, generated with S2 amplification primer will remain single-stranded (FIG. 12b).

### DETAILED DESCRIPTION OF THE INVENTION

The present invention provides a microsensor device and method for the detection of target analytes. In addition the invention provides a multi-component device for the simultaneous detection of multiple analytes of interest. The microsensor device may include multiple chambers for independent measurement or detection of target analytes.

The apparatus of the invention also includes a surface acoustic wave sensor, or a plurality of surface acoustic wave sensors, for the detection of one or a plurality of target analytes. Briefly, a surface acoustic wave sensor comprises a piezoelectric layer, or piezoelectric substrate, and input and output transducer(s). A surface acoustic wave is generated within the piezoelectric layer by an electronic input signal applied to the input transducer. The wave propagates along the piezoelectric layer and is electrically detected by the output transducer. As described in more detail below, binding events that alter the surface of the surface acoustic wave sensor can be detected as a change in a property of the propagating surface acoustic wave. Suitable surface acoustic wave sensors are described in U.S. Pat. Nos. 5,130,257; 5,283,037; and 5,306,644; F. Josse, et. al. “Guided Shear Horizontal Surface Acoustic Wave Sensors for Chemical and Biochemical Detection in Liquids,” Anal. Chem. 2001, 73, 5937; and W. Welsch, et. al., “Development of a Surface Acoustic Wave Immunosensor,” Anal. Chem. 1996, 68, 2000-2004; all of which are hereby expressly incorporated by reference.

By ‘surface acoustic wave sensor’, or ‘surface acoustic wave device’ herein is meant any device that operates substantially in the manner described above. In some embodiments, ‘surface acoustic wave sensor’ refers to both surface transverse wave devices, where the surface displacement is perpendicular to the direction of propagation and parallel to the device surface, as well as surface acoustic wave sensors where at least a portion of the surface displacement is perpendicular to the device surface. While surface transverse wave devices generally have better sensitivity in a fluid, it has been shown that sufficient sensitivity may also be achieved when a portion of the surface displacement is perpendicular to the device surface. See, for example, M. Rapp, et. al. “Modification of Commercially Available LOW-LOSS SAW devices towards an immunosensor for in situ Measurements in Water”1995 IEEE International Ultrasonics Symposium, Nov. 7-10, 1995, Seattle, Wash.; and N. Barie, et. al., “Covalent bound sensing layers on surface acoustic wave biosensors,” Biosensors & Bioelectronics 16 (2001) 979; all of which are expressly incorporated herein by reference.

Accordingly, the surface acoustic wave sensors of the present invention comprise a piezoelectric layer, or piezoelectric substrate. The piezoelectric substrate may be made from quartz, lithium niobate (LiNbO₃), or any other piezoelectric material. The cut of the piezoelectric substrate relative to its crystal structure should be such that acoustic waves are trapped at the surface and the desired direction of material displacement relative to the surface and to the propagating wave (as described above) is achieved.

The input and output transducers are preferably interdigital transducers. Generally, there are two interdigital transducers; each of the input and output transducers comprises two electrodes, such that an applied voltage difference between the two electrodes of the input transducer results in the generation of a surface acoustic wave in the piezoelectric substrate. The electrodes generally may comprise any conductive material, with aluminum or gold being preferred.

In an alternative embodiment there is a single interdigital transducer. In this embodiment the single interdigital transducer, serves both as both an input and output transducer. In embodiments employing a single interdigital transducer acting as both input and output transducer, a reflector structure is generally provided to generate one or more resonances within the SAW sensor. The reflector structure may, for example, be a thin film grating. The grating may comprise aluminum, or another conductive material. The generated resonances can be detected, for example, by measuring the power dissipated at the single transducer. One or more binding events alter these resonances, allowing the binding events to be detected. An example of a sensor and technique according to this embodiment is generally described in U.S. Pat. No. 5,846,708, hereby incorporated by reference. As described below, other electronics and/or circuitry may similarly be utilized in an embodiment employing a SAW sensor having only one interdigitated transducer.

With gold electrodes, a DNA probe molecule may be attached using a SH group on the 5′ of the DNA using self-assembled monolayers as known in the art and described, for example, in K. Vijayamohanan et al. “Self-assembled monolayers as a tunable platform for biosensor applications,” Biosensors & Bioelectronics 17 (2002) 1-12 and George M. Whitesides et al. “Array of Self-Assembled Monolayers for studing inhibition of Bacterial Adhesion.” Anal Chem 2002, 74, 1805-1810, both of which are hereby incorporated by reference.

In preferred embodiments, surface acoustic wave sensors of the present invention comprise a polymer layer covering all or a portion of the input and output transducers and the piezoelectric surface. The polymer layer generally serves two purposes. The first is to shield the input and output transducers from the sample fluid. The second is to act as a waveguide to direct the propagating surface acoustic wave. The polymer layer may comprise any material that serves the above two purposes. In preferred embodiments, the polymer layer comprises polyimide.

The surface acoustic wave sensor further comprises one or more binding ligands attached to at least a portion of the sensor surface for binding a target analyte, discussed further below. The attachment can be either covalent or non-covalent.

Reference sensors according to the present invention are generally structured as described above, but are designed to generate a reference output signal corresponding to a baseline output signal indicative of the absence of binding events. Accordingly, binding ligands may be immobilized on the reference sensor and a buffer solution, containing no suitable target analytes for binding to the reference sensor, is applied to the reference sensor. Alternatively, reference sensors according to the present invention may not comprise binding ligands, or may comprise binding ligands which do not form a complex with a desired target analyte.

Reference sensors may be substantially integrated with one or more active sensors. For example, a reference sensor may be formed on the same substrate as an active sensor. A reference sensor may further be formed on the same piezoelectric layer as an active sensor. Alternatively, a reference sensor may be formed independent of any active sensors, and merely operatively associated with an active sensor through electronic detection circuitry. Surface acoustic wave filters are available commercially. For example, SAW filters manufactured by MuRaTa, type SAF380F are particularly preferred for adaptation for use in the present invention.

Generally, in the case of a single, active SAW sensor, an electronic input signal having an amplitude, frequency, and phase is applied to an input transducer. An ouput signal having a second amplitude, frequency, and phase is detected at an output transducer. A binding event can be detected by monitoring—continuously or at predefined times—the ouput signal before and after binding. A shift in the amplitude, frequency, or phase of the output signal may be indicative of a binding event.

The above detection procedure may be extended in other embodiments to cases where more that one sensor is employed.

In other embodiments, one or more sensor output signals may be compared to indicate a binding event. A discussed above, one or more output signals from active devices may further be compared to an output signal from a reference sensor. When a similar input signal had been applied to an active and a reference sensor, a direct comparison between properties of output signals may reliably indicate a binding event.

In other embodiments, those having skill in the art will appreciate that a variety of signal processing techniques may be employed to design one or more input signals and process output signals obtained from one or more sensors for reliable identification of a binding event.

Accordingly, appropriate circuitry may be provided in association with the input and output transducers to generate input signals, detect, and compare output signals. This includes, for example, differential amplifiers, oscillators, oscillator circuits employing the SAW sensor as a frequency determination element, signal generators, network analyzers, voltmeters, multimeters, as well as other amplifying, frequency detecting, conditioning, control, and differential circuitry as known in the art. As appropriate, circuitry may be integrated with one or more sensors, or merely operatively associated with a sensor.

Preferably, the surface acoustic wave sensors of the invention are positioned in a channel or chamber. The channel or chamber has inlet or outlet ports which allow for the introduction of samples into the channel or chamber for analysis of target samples. In one embodiment, the sample may be separated, for example, into different channels or chambers for separate analysis. That is, in one embodiment multiple samples can be analyzed simultaneously. In an alternative embodiment multiple target analytes can be analyzed from a single sample. That is, a plurality of discrete microsensors may be contained within a single chamber. In this embodiment the individual microsensors may be used to detect discrete target analytes from a single sample.

Accordingly, the surface acoustic wave sensor of the invention is used to detect target analytes in samples. By “target analyte” or “analyte” or grammatical equivalents herein is meant any molecule, compound or particle to be detected. As outlined below, target analytes preferably bind to binding ligands, as is more fully described herein. Preferably the binding ligands are immobilized to a surface of the surface acoustic wave sensor. As will be appreciated by those in the art, a large number of analytes may be detected using the present methods; basically, any target analyte, for which a binding ligand exists, may be detected using the methods and apparatus of the invention.

Suitable analytes include organic and inorganic molecules, including biomolecules. In a preferred embodiment, the analyte may be an environmental pollutant (including heavy metals, pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc)(detection of antigen antibody interactions are described in U.S. Pat. Nos. 4,236,893, 4,242,096, and 4,314,821, all of which are expressly incorporated herein by reference); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the target analyte and binding ligands are nucleic acids. By “nucleic acid” or “oligonucleotide” or grammatical equivalents herein means at least two nucleotides covalently linked together.

In a preferred embodiment, the present invention provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. Target sequences also include the result or product of an amplification reaction, i.e. amplicons.

A nucleic acid of the present invention will generally contain phosphodiester bonds, although in some cases, as outlined below, nucleic acid analogs that may have alternate backbones may be used. Preferably, the nucleic acid target analyte is a polynucleotide. Nucleic acid analogs are preferably used, if at all, as immobilized probes (binding ligand) on the surface of a microsensor. Such nucleic acid analytes have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) ppl69-176). In addition, locked nucleic acids (LNA) find use in the invention. LNA are described in more detail in Wengel el al.; J. Org Chem 63; 10035-9 1998, which is expressly incorporated herein by reference. Several nucleic acid analogs are described in Rawls, C & E News Jun. 2, 1997 page 35. All of these references are hereby expressly incorporated by reference. These modifications of the ribose-phosphate backbone may be done to facilitate the addition of labels or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids whether a target nucleic acid, probe or elongation product, for example of a polymerase or a ligase, may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As is outlined more fully below, probes (including amplification primers) are made to hybridize to target sequences to determine the presence or absence of the target sequence in a sample. Generally speaking, this term will be understood by those skilled in the art.

The target sequence may also be comprised of different target domains, for example, in “sandwich” type assays as outlined below, a first target domain of the sample target sequence may hybridize to an immobilized probe or primer on a microsensor, i.e. SAW sensor as described herein, and a second target domain may hybridize to a solution-phase probe or primer. In addition, the target domains may be adjacent (i.e. contiguous) or separated. For example, when ligation techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below. In such cases, at least one of the primers is immobilized on the surface of a microsensor and a ligase is used to covalently join the probe.

In another preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. As discussed below, when the protein is used as a binding ligand, it may be desirable to utilize protein analogs to retard degradation by sample contaminants.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc.

Accordingly, the present invention provides a single or multi-component devices for the detection of target analytes. As noted above, the device includes a detection channel or chamber that includes at least one active SAW sensor and may preferably contain at least 4, 5, 10, 20, 30, 40, 50 or 100 active SAW sensors. In a preferred embodiment the chamber includes at least 100 SAW sensors. As described herein, the SAW sensors are coupled to a detector.

In one embodiment the device includes a single channel or chamber for the amplification and detection of target nucleic acids. Alternatively, the device may comprise more than one channel or chamber; for example, there may be a “sample treatment” or “sample preparation” channels or chambers that interfaces with a separate “detection” channel or chamber. By “channel” is meant a path or trough through which a sample flows, generally between chambers, although in some embodiments reactions can occur in the channels themselves. By “chamber” is meant a closed or closeable portion of the microfluidic device in which samples are manipulated and/or detected. While much of the discussion below emphasizes reactions occurring in chambers, it is appreciated that any of the reactions or manipulations also can occur in channels.

Generally, when nucleic acids are to be detected and nucleic acids serve as the probes or primers, two general schemes find use in the invention. In one embodiment the target analyte is amplified to produce amplicons. Amplicons are then detected with the microsensor. In another embodiment, the target analyte hybridizes with the probe or primer immobilized on the microsensor. The probe or primer is modified and the modification, which generally includes a change in the mass of the probe or primer, is detected. As one of skill in the art appreciates, “target analytes” can include both targets from samples or products of an amplification reaction, i.e. amplicons. That is, amplicons can serve as target analytes. The immobilized probe can then be modified as a result of hybridization with the amplicons. Alternatively, specific hybridization of a target with the immobilized probe on the sensor results in a detectable change in an actual or differential sensor output signal.

As noted previously, detection of target analytes can occur by hybridization of a target to a probe immobilized on the surface of a substrate. Detection also can occur by detecting a modification of the immobilized probe or primer. This results in the formation of a “modified primer”. While there are a variety of types of modifications, generally modifications that find use in the present invention are those that result in a change in mass of the immobilized probe or primer. That is, in general the probe or primer will be modified by extension such as by a DNA polymerase or ligase. Sandwich assays also find use in detection of target analytes.

As discussed herein, it should be noted that the sandwich assays can be used for the detection of primary target sequences (e.g. from a patient sample), or as a method to detect the product of an amplification reaction as outlined above; thus for example, any of the newly synthesized strands outlined above, for example using PCR, LCR, NASBA, SDA, etc., may be used as the “target sequence” in a sandwich assay. Sandwich assays are described in U.S. S. No. 60/073,011 and in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In addition these target sequences can be used as templates for other assays that result in modification of the immobilized primers.

Single Base Extension (SBE) is an extension assay that results in the incorporation of a nucleotide into a primer sequence when the primer sequence is complementary to or hybridized with a target sequence. The nucleotide incorporated into the primer is complementary to the nucleotide at the corresponding position of the target nucleic acid. Accordingly, the immobilized primer is extended, i.e. modified, and is detected by the device of the invention. As such, detection of a change in the immobilized primer is an indication of the presence of the target analyte.

In addition, just as primer extension can be detected on a SAW sensor, sequencing can be performed on a SAW sensor. When primer extension is performed, the detector detects an increase in mass that is indicative of the addition of a nucleotide. When sequencing is performed, each nucleotide that is added to the primer is detected on the SAW sensor. That is, the detector detects which nucleotide is added to the primer. As such, the sequence of the target can be obtained. In some embodiments, nucleotides are added to the primer extension reaction one at a time. In this embodiment, upon detecting an increase in mass on the SAW sensor, the sequence also is determined. In an alternative embodiment, the nucleotides are tagged or labeled with particles, i.e. gold particles, of characteristic mass. That is, each of the nucleotides is tagged with a label of discrete mass that is indicative of the particular nucleotide.

In some embodiments, when the tag prevents subsequent primer extension, the tagged nucleotides are added in combination with untagged nucleotides. This way, a population of primers will be extended with tagged nucleotides while a population will be extended with untagged nucleotides that are available for additional extension. In this way, the sequence of the target nucleic acid is obtained.

Oligonucleotide-ligation assay is an extension of PCR-based screening that uses an ELISA-based assay (OLA, Nickerson et al., Proc. Natl. Acad. Sci. USA 87:8923, 1990) to detect the PCR products that contain the target sequence. Briefly, the OLA employs two adjacent oligonucleotides: a “reporter” probe and an “anchor” probe. The two oligonucleotides are annealed to target DNA and, if there is perfect complementarity, the two probes are ligated by a DNA ligase. The ligated probe is then captured by the probe on the SAW sensor.

Alternatively, one of the OLA primers is immobilized on the microsensor. Upon ligation, the mass on the microsensor is increased. The mass increase is detected as an indication of the presence of the target analyte.

In this and other embodiments, a heating and/or cooling module may be used, that is either part of the reaction chamber or separate but can be brought into spatial proximity to the reaction module. Suitable heating modules are described in U.S. Pat. Nos. 5,498,392 and 5,587,128, and WO 97/16561, incorporated by reference, and may comprise electrical resistance heaters, pulsed lasers or other sources of electromagnetic energy directed to the reaction chamber. It should also be noted that when heating elements are used, it may be desirable to have the reaction chamber be relatively shallow, to facilitate heat transfer; see U.S. Pat. No. 5,587,128.

In one embodiment, the devices of the invention includes a separate detection module. That is, when the reaction channel or chamber does not include the microsensors, a separate detection channel or chamber is needed. It should be noted that the following discussion of detection modules is applicable to the microsensor when the microsensors are found in the reaction channel or chamber.

Accordingly, the present invention is directed to methods and compositions useful in the detection of biological target analyte species such as nucleic acids and proteins. In general, the detection module is based on binding partners or bioactive agents attached to microsensors as described herein.

That is, each microsensor comprises a bioactive agent. By “candidate bioactive agent” or “bioactive agent” or “chemical functionality” or “binding ligand” herein is meant any molecule, e.g., protein, oligopeptide, small organic molecule, coordination complex, polysaccharide, polynucleotide, etc. which can be attached to a microsensor. Preferred bioactive agents include biomolecules including peptides, nucleic acids, saccharides, fatty acids, steroids, purines, pyrimidines, derivatives, structural analogs or combinations thereof. Particularly preferred are nucleic acids and proteins.

In one preferred embodiment, the bioactive agents are naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

In a preferred embodiment, the bioactive agents are peptides of from about 5 to about 30 amino acids, with from about 5 to about 20 amino acids being preferred, and from about 7 to about 15 being particularly preferred.

In a preferred embodiment, the bioactive agents are nucleic acids as defined above (generally called “probe nucleic acids”, “primers” or “candidate probes” herein). As described above generally for proteins, nucleic acid bioactive agents may be naturally occurring nucleic acids, random nucleic acids, or “biased” random nucleic acids. For example, digests of procaryotic or eukaryotic genomes may be used as is outlined above for proteins.

When the bioactive agents are nucleic acids, they are designed to be substantially complementary to target sequences. As noted above, the term “target sequence” or grammatical equivalents herein means a nucleic acid sequence on a single strand of nucleic acid.

A probe nucleic acid (also referred to herein as a primer nucleic acid) is then contacted with the target sequence to form a hybridization complex. Generally, the probe nucleic acid is immobilized on the surface of a microsensor, i.e. SAW sensor. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The size of the probe or primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on what is required for detection and/or amplification as is discussed below.

In a preferred embodiment, each microsensor comprises a single type of bioactive agent, although a plurality of individual bioactive agents are preferably attached to each microsensor, as described herein. In addition, as described above, the microsensor is in communication with a detector such that the presence of the target analyte can be determined.

In a preferred embodiment, the devices of the invention include a reaction module. This can include either physical, chemical or biological alteration of one or more sample components. Alternatively, it may include a reaction module wherein the target analyte alters a second moiety that can then be detected; for example, if the target analyte is an enzyme, the reaction chamber may comprise a substrate that upon modification by the target analyte, can then be detected by binding to a microsensor. In this embodiment, the reaction module may contain the necessary reagents, or they may be stored in a storage module and pumped as outlined herein to the reaction module as needed.

Alternatively, the target analyte serves as a substrate for an enzymatic reaction such as a polymerase or ligase extension reaction, but the target itself is not altered or consumed. Rather, the immobilized probe or primer in the microsensor is modified in a template or target analyte dependent manner.

In a preferred embodiment, the reaction module includes a chamber for the chemical modification of all or part of the sample before or during analyte detection. That is, in one embodiment there is a separate reaction module and a separate detection module. In an alternative embodiment the reaction occurs in the detection module. This allows for simultaneous modification and detection of analytes.

Chemical modifications include, but are not limited to chemical cleavage of sample components (CNBr cleavage of proteins, etc.) or chemical cross-linking. PCT US97/07880, hereby incorporated by reference, lists a large number of possible chemical reactions that can be performed in the devices of the invention, including amide formation, acylation, alkylation, reductive amination, Mitsunobu, Diels Alder and Mannich reactions, Suzuki and Stille coupling, etc. Similarly, U.S. Pat. Nos. 5,616,464 and 5,767,259 describe a variation of ligation chain reaction (LCR; sometimes also referred to as oligonucleotide ligation amplification or OLA) that utilizes a “chemical ligation” of sorts.

In a preferred embodiment, the reaction module includes a chamber for the biological alteration of all or part of the sample before or during analyte detection. For example, enzymatic processes including nucleic acid amplification and other nucleic acid modifications including ligation, cleavage, circularization, supercoiling, methylation, acetylation; hydrolysis of sample components or the hydrolysis of substrates by a target enzyme, the addition or removal of detectable labels, the addition or removal of phosphate groups, protein modification (acylation, glycosylation, addition of lipids, carbohydrates, etc.), the synthesis/modification of small molecules, etc.

Alternatively, the modification or alteration may occur in the immobilized primer as a result of hybridization with the target molecule.

In a preferred embodiment, the target analyte is a nucleic acid and the biological reaction chamber allows amplification of the target nucleic acid. Suitable amplification techniques include polymerase chain reaction (PCR), reverse transcriptse PCR (RT-PCR), ligase chain reaction (LCR), and Invader™ technology. Techniques utilizing these methods are well known in the art. In this embodiment, the reaction reagents generally comprise at least one enzyme (generally polymerase), primers, and nucleoside triphosphates as needed. As described herein, the amplification reactions can occur in a chamber or channel separate from the detection chamber. Alternatively, the amplification can occur in the detection chamber. As amplification proceeds, the amplicons hybridize to the immobilized probe on the microsensor in the detection chamber resulting in a detectable change in a property of the microsensor as outlined herein.

Alternatively, the amplicons serve as templates for subsequent reactions that result in a modification of the immobilized primer. Such modifications are discussed more fully below and include primer extension that results in lengthening the primer. Also, the primer can be ligated to another probe or primer such that the immobilized primer is lengthened.

General techniques for nucleic acid amplification are discussed below. In most cases, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques such as the use of extra probes or nucleic acid binding proteins may also be used. In one embodiment isothermal amplification is preferred.

In addition, the different amplification techniques may have further requirements of the primers, as is more fully described below.

Once the hybridization complex between the primer and the target sequence has been formed, an enzyme, sometimes termed an “amplification enzyme”, is used to modify the immobilized primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides or oligonucleotides are added to the primer to extend its length.

In some embodiments, once the enzyme has modified the primer to form a modified primer, the hybridization complex is disassociated. By “modified primer” is meant a primer that has been changed or altered in a detectable manner. Generally a modified primer is lengthened by the addition of at least one nucleotide.

In an alternative embodiment proteins are the target molecules and are detected by protein affinity agents that include a nucleic acid to be amplified. By “affinity agent” is meant a molecule that binds with high affinity to the target protein. Affinity agents can include, but are not limited to aptamers, antibodies, ligands, adapter proteins, lectins, and the like. In this embodiment the affinity agent is coupled to a nucleic acid.

After a binding reaction between the protein target and the affinity agent, the unbound affinity agents are removed. Agents can be removed by methods as known in the art, such as by washing. In this embodiment it is preferable for the complexes to be immobilized so that the unbound molecules can be washed away. Once removed, the nucleic acids are amplified and the resulting amplicons are detected by hybridization to immobilized probes on the SAW sensor as described herein. Alternatively the nucleic acids are not themselves amplified, but serve to hybridize with a circular probe. The circular probe is a template for Rolling Circle Amplification. This is described in more detail in Nature Biotechnology, April, 2002, vol. 20, pp359-365, which is expressly incorporated herein by reference. Following the Rolling Circle Amplification, the amplicons again are detected on the SAW sensor as described herein.

Thus, for both protein detection and nucleic acid detection amplification of nucleic acids may occur prior to detection of the target molecule. During amplification generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

In one embodiment, after a suitable time or amplification, the amplicon is moved to a detection module and incorporated into a hybridization complex with a probe immobilized on the surface of a microsensor, as is more fully outlined below. The hybridization complex is attached to a microsensor and detected, as is described below.

In an alternative embodiment, amplification occurs in the detection chamber (described more fully below). That is, amplification and detection occur in the same chamber. In one embodiment amplification proceeds by using at least two solution phase primers. Following amplification, amplicons hybridize with probes or primers immobilized on the surface of the microsensor to form hybridization complexes. Upon hybridization with the immobilized probe, the presence of the target analyte is detected. In a preferred embodiment, the hybridization complex is used as a template for further reactions that result in the modification of the immobilized probe. Such reactions include extension reactions such as single base extension (SBE), template dependent nucleic acid synthesis or the oligonucleotide ligation assay (OLA) described in more detail herein.

In an alternative embodiment amplification and primer extension proceeds by the use of a solution-phase primer and a primer immobilized on the surface of the microsensor.

In yet another alternative embodiment, amplification proceeds by the use of primer pairs immobilized on the surface of a microsensor. That is, both amplification primers are immobilized on the surface of the microsensor. As such, upon amplification of the target analyte, the amplicons also are immobilized on the surface of the microsensor.

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA) and the ligase chain reaction (LCR).

In a preferred embodiment, the target amplification technique is PCR. The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtration”, among others.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostable polymerase such as Taq I polymerase and thermal cycling. In an alternative embodiment isothermal amplification is used.

Accordingly, the PCR reaction requires at least one PCR primer and a polymerase. Mesoscale PCR devices are described in U.S. Pat. Nos. 5,498,392 and 5,587,128, and WO 97/16561, incorporated by reference.

In a preferred embodiment the amplification is RT-PCR. Preferably the reaction includes either two-step RT-PCR or solid phase RT-PCR. In this embodiment RT-PCR can be performed using either solution phase primers or immobilized primers as described above. In this embodiment mRNA is reverse transcribed to CDNA and PCR is conducted by using DNA polymerase. Again PCR primers can be solution-phase or immobilized as described above.

In an additional preferred embodiment, re-amplification of CDNA (multiple-PCR system) is performed. cDNA synthesized from mRNA can be used more than once. Preferably, the cDNA is immobilized as this increases the stability of the cDNA. This allows reamplification of the same immobilized CDNA such that different or the same target sequences can be amplified multiple times. As noted above, amplification can use solution-phase primers or immobilized primers and detection of amplicons proceeds following hybridization of amplicons to the probe immobilized on the microsensor.

In a preferred embodiment the RT-PCR amplification is a high throughput RT-PCR system.

In a preferred embodiment, the amplification technique is LCR. The method can be run in two different ways; in a first embodiment, only one strand of a target sequence is used as a template for ligation; alternatively, both strands may be used. See generally U.S. Pat. Nos. 5,185,243 and 5,573,907; EP 0 320 308 B1; EP 0 336 731 B 1; EP 0 439 182 B1; WO 90/01069; WO 89/12696; and WO 89/09835, and U.S. S. Nos. 60/078,102 and 60/073,011, all of which are incorporated by reference.

In a preferred amplification embodiment, the single-stranded target sequence comprises a first target domain and a second target domain. A first LCR primer and a second LCR primer nucleic acids are added, that are substantially complementary to its respective target domain and thus will hybridize to the target domains. These target domains may be directly adjacent, i.e. contiguous, or separated by a number of nucleotides. If they are non-contiguous, nucleotides are added along with means to join nucleotides, such as a polymerase, that will add the nucleotides to one of the primers. The two LCR primers are then covalently attached, for example using a ligase enzyme such as is known in the art. This forms a first hybridization complex comprising the ligated probe and the target sequence. This hybridization complex is then denatured (disassociated), and the process is repeated to generate a pool of ligated probes, i.e. amplicons. The ligated probes or amplicons are then detected with the probe immobilized on the microsensor.

In a preferred embodiment, LCR is done for two strands of a double-stranded target sequence. The target sequence is denatured, and two sets of primers are added: one set as outlined above for one strand of the target, and a separate set (i.e. third and fourth primer nucleic acids) for the other strand of the target. In a preferred embodiment, the first and third primers will hybridize, and the second and fourth primers will hybridize, such that amplification can occur. That is, when the first and second primers have been attached, the ligated product can now be used as a template, in addition to the second target sequence, for the attachment of the third and fourth primers. Similarly, the ligated third and fourth products will serve as a template for the attachment of the first and second primers, in addition to the first target strand. In this way, an exponential, rather than just a linear, amplification can occur.

Again, as outlined above, the detection of the LCR products can occur directly, in the case where one or both of the primers simply hybridize with a primer immobilized on the microsensor; hybridization is detected as described herein. Alternatively, detection of LCR products can occur indirectly using sandwich assays, through the use of additional probes; that is, the ligated products can serve as target sequences, and detection proceeds via hybridization to probes or primers immobilized on the surface of the microsensor.

In addition, the device may include other modules such as sample preparation chambers. In this embodiment, a crude sample is added to the sample treatment channel or chamber and is manipulated to prepare the sample for detection. The manipulated sample is removed from the sample treatment channel or chamber and added to the detection chamber. There may be additional functional elements into which the device fits; for example, a heating element may be placed in contact with the sample channel or chamber to effect reactions such as PCR. In some cases, a portion of the device may be removable; for example, the sample chamber may have a detachable detection chamber, such that the entire sample chamber is not contacted with the detection apparatus. See for example U.S. Pat. No. 5,603,351 and PCT US96/17116, hereby incorporated by reference.

In addition to different channels or chambers, the device may also include one or more flow cells or flow channels allowing sample movement between chambers. In addition to flow channels, there also may be inlet ports and outlet ports separating chambers. Such ports allow for samples to be contained in different chambers without cross-contamination.

In some embodiments the device also includes a pump mechanism that hydrodynamically pumps the samples through the device. Alternatively a vacuum device is used.

In a preferred embodiment, the microfluidic device can be made from a wide variety of materials, including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, etc.

The microfluidic devices of the invention can be made in a variety of ways, as will be appreciated by those in the art. See for example WO96/39260, directed to the formation of fluid-tight electrical conduits; U.S. Pat. No. 5,747,169, directed to sealing; and EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351, all of which are hereby incorporated by reference. Suitable fabrication techniques again will depend on the choice of substrate, but preferred methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding, and bonding techniques (see U.S. Pat. No. 5,747,169, hereby incorporated by reference). In addition, there are printing techniques for the creation of desired fluid guiding pathways; that is, patterns of printed material can permit directional fluid transport.

In a preferred embodiment, the device is configured for handling a single sample that may contain a plurality of target analytes. That is, a single sample is added to the device and the sample may either be aliquoted for parallel processing for detection of the analytes or the sample may be processed serially, with individual targets being detected in a serial fashion.

In a preferred embodiment, the solid substrate is configured for handling multiple samples, each of which may contain one or more target analytes. In general, in this embodiment, each sample is handled individually; that is, the manipulations and analyses are done in parallel, with preferably no contact or contamination between them. Alternatively, there may be some steps in common; for example, it may be desirable to process different samples separately but detect all of the target analytes on a single detection array, as described below.

Thus, the multi-chamber devices of the invention include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of different channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used; similarly, longer lengths for separation purposes may also be desirable.

In general, the microfluidic devices of the invention are generally referred to as “mesoscale” devices. The devices herein are typically designed on a scale suitable to analyze microvolumes, although in some embodiments large samples (e.g. cc's of sample) may be reduced in the device to a small volume for subsequent analysis. That is, “mesoscale” as used herein refers to chambers and microchannels that have cross-sectional dimensions on the order of 0.1 μm to 500 μm. The mesoscale flow channels and wells have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. However, for many applications, larger dimensions on the scale of millimeters may be used. Similarly, chambers in the substrates often will have larger dimensions, on the scale of a few millimeters.

In addition to the flow channel system, the devices of the invention may be configured to include one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell concentration, cell separation or capture, cell fusion, cell growth, etc.); separation modules, for example, for electrophoresis, gel filtration, sedimentation, etc.); reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), chemical, physical or enzymatic cleavage or alteration of the target analyte, or chemical modification of the target; fluid pumps; fluid valves; heating modules; storage modules for assay reagents; mixing chambers; and detection modules.

In a preferred embodiment, the devices of the invention include at least one sample inlet port for the introduction of the sample to the device. This may be part of or separate from a sample introduction or collection module; that is, the sample may be directly fed in from the sample inlet port to a separation chamber, or it may be pretreated in a sample collection well or chamber. Alternatively, for example, when there is a single chamber, the sample inlet port may be configured such that samples are introduced into the single chamber for amplification and/or detection.

In a preferred embodiment, the devices of the invention include a sample collection module, which can be used to concentrate or enrich the sample if required; for example, see U.S. Pat. No. 5,770,029, including the discussion of enrichment channels and enrichment means.

In a preferred embodiment, the devices of the invention include a cell handling module. This is of particular use when the sample comprises cells that either contain the target analyte or that are removed in order to detect the target analyte. Thus, for example, the detection of particular antibodies in blood can require the removal of the blood cells for efficient analysis, or the cells must be lysed prior to detection. In this context, “cells” include viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In a preferred embodiment, the cell handling module includes a cell lysis module. As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In a preferred embodiment, the cell lysis module comprises a cell lysing agent, such as detergents, NaOH, enzymes, proteinase K, guanidinium HCL, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample. Temperature or mixing may also be applied.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B1, incorporated by reference.

In one embodiment of sample preparation, cells are placed or distributed on a filter membrane evenly and a lysis buffer is passed through the cell layer on the filter membrane without mechanical homogenization of the cells. This can be performed in a sample preparation chamber as described above. Alternatively, it may be performed prior to addition of the sample to the chamber.

In the above, the cell lysate can be passed through the membrane of the filter plate with the aid of force generated by means of centrifugation, vacuum, or positive pressure. The filter or membrane of the filter plate includes, but is not limited to, glass fiber, polypropylene or polyolefine mesh, wool, and other membranes which have a pore size such that target cells can be trapped without any leakage of cells from the membrane, but cytosolic mRNA can pass through. For example, using glass fiber (Grade 934AH, Cambridge Technology, Inc. Watertown, Mass.) or Whatman GFIF grade glass fiber membrane, most of cultured cells and blood leukocyte can be trapped. In the above, glass fiber plates are preferable.

The lysis buffer may include a detergent for dissolving cell membranes, RNase inhibitor for inhibiting RNase activity or deactivating or destroying RNase, and pH control agent and salt for hybridization. The isolated target sample can then be analyzed as described herein

Accordingly, a rapid, inexpensive, high throughput, and easily automated system can be realized.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample population. These binding moieties may be immobilized either on the surface of the module or on a particle trapped within the module by physical absorption or by covalent attachment. Suitable binding moieties will depend on the cell type to be isolated or removed, and generally includes antibodies and other binding ligands, such as ligands for cell surface receptors, etc. Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size or shape. This can be done in a variety of ways, including protrusions from the surface that allow size exclusion, a series of narrowing channels, or a diafiltration type setup.

In a preferred embodiment, the cell handling module includes a cell removal module. This may be used when the sample contains cells that are not required in the assay. Generally, cell removal will be done on the basis of size exclusion as for “sieving”, above, with channels exiting the cell handling module that are too small for the cells; filtration and centrifugation may also be done.

In a preferred embodiment, the cell handling module includes a cell concentration module. As will be appreciated by those in the art, this is done using “sieving” methods, for example to concentrate the cells from a large volume of sample fluid prior to lysis, or centrifugation.

In a preferred embodiment, the devices of the invention include a separation module. Separation in this context means that at least one component of the sample is separated from other components of the sample. This can comprise the separation or isolation of the target analyte, or the removal of contaminants that interfere with the analysis of the target analyte, depending on the assay.

In a preferred embodiment, the separation module includes chromatographic-type separation media such as absorptive phase materials, including, but not limited to reverse phase materials (C₈ or C₁₈ coated particles, etc.), ion-exchange materials, affinity chromatography materials such as binding ligands, etc. See U.S. Pat. No. 5,770,029.

In a preferred embodiment, the separation module utilizes binding ligands, as is generally outlined herein for cell separation or analyte detection.

When the sample component bound by the binding ligand is the target analyte, it may be released for detection purposes if necessary, using any number of known techniques, depending on the strength of the binding interaction, including changes in pH, salt concentration, temperature, etc. or the addition of competing ligands, etc.

In a preferred embodiment, the separation module includes an electrophoresis module, as is generally described in U.S. Pat. Nos. 5,770,029; 5,126,022; 5,631,337; 5,569,364; 5,750,015, and 5,135,627, all of which are hereby incorporated by reference. In electrophoresis, molecules are primarily separated by different electrophoretic mobilities caused by their different molecular size, shape and/or charge. Microcapillary tubes have recently been used for use in microcapillary gel electrophoresis (high performance capillary electrophoresis (HPCE)). One advantage of HPCE is that the heat resulting from the applied electric field is efficiently dissipated due to the high surface area, thus allowing fast separation. The electrophoresis module serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the electrophoresis module can take on a variety of forms, and generally comprises an electrophoretic microchannel and associated electrodes to apply an electric field to the electrophoretic microchannel. Waste fluid outlets and fluid reservoirs are present as required.

The electrodes comprise pairs of electrodes, either a single pair, or, as described in U.S. Pat. Nos. 5,126,022 and 5,750,015, a plurality of pairs. Single pairs generally have one electrode at each end of the electrophoretic pathway. Multiple electrode pairs may be used to precisely control the movement of sample components, such that the sample components may be continuously subjected to a plurality of electric fields either simultaneously or sequentially. Such a system is outlined in 5,858,195, incorporated herein by reference

In a preferred embodiment, electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

In a preferred embodiment, the devices of the invention include at least one fluid pump. Pumps generally fall into two categories: “on chip” and “off chip”; that is, the pumps (generally syringe pumps or electrode based pumps) can be contained within the device itself, or they can be contained on an apparatus into which the device fits, such that alignment occurs of the required flow channels to allow pumping of fluids.

In a preferred embodiment, the devices of the invention include at least one fluid valve that can control the flow of fluid into or out of a module of the device. A variety of valves are known in the art. For example, in one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Preferably, capillary barriers include a dam that raises the vertical height of the channel immediately before the opening into a larger space such a chamber. In addition, as described in U.S. Pat. No. 5,858,195, incorporated herein by reference, a type of “virtual valve” can be used.

In a preferred embodiment, the devices of the invention include sealing ports, to allow the introduction of fluids, including samples, into any of the modules of the invention, with subsequent closure of the port to avoid the loss of the sample.

Once made, the device of the invention finds use in a variety of applications. Preferred applications include forensics, mutation detection, microorganism or pathogen detection and the like.

As to forensics, the identification of individuals at the level of DNA sequence variation offers a number of practical advantages over such conventional criteria as fingerprints, blood type, or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. Two types of probes are now in use for DNA fingerprinting by DNA blots. Polymorphic minisatellite DNA probes identify multiple DNA sequences, each present in variable forms in different individuals, thus generating patterns that are complex and highly variable between individuals. VNTR probes identify single sequences in the genome, but these sequences may be present in up to 30 different forms in the human population as distinguished by the size of the identified fragments. The probability that unrelated individuals will have identical hybridization patterns for multiple VNTR or minisatellite probes is very low. Much less tissue than that required for DNA blots, even single hairs, provides sufficient DNA for a PCR-based analysis of genetic markers. Also, partially degraded tissue may be used for analysis since only small DNA fragments are needed. Forensic DNA analyses will eventually be carried out with polymorphic DNA sequences that can be studied by simple automatable assays such as OLA. For example, the analysis of 22 separate gene sequences, each one present in two different forms in the population, could generate 1010 different outcomes, permitting the unique identification of human individuals. That is, the unique pattern of mass increases as a result of detecting unique genes, exon/intron boundaries, SNPs, mRNA and the like results in the unique identification of an individual.

In another preferred embodiment the device finds use in tumor diagnostics. The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such s point mutations (as in the c-K-ras oncogene in bladder carcinoma and in colorectal tumors), promoter induction, gene amplification (as in the N-myc oncogene in the case of neuroblastoma) or the rearrangement of chromosomes (as in the translocation of the c-abl oncogene from chromosome 9 to chromosome 22 in the case of chronic myeloid leukemia). Each of the activation processes leads, in conjunction with additional degenerative processes, to an increased and uncontrolled cell growth. The so-called “recessive oncogenes” which must be inactivated for the fonmation of a tumor (as in the retinobiastoma (Rb gene and the osteosarcoma can also be detected with the help of DNA probes. Using probes against immunoglobulin genes and against T-cell receptor genes, the detection of B-cell lymphomas and lymphoblastic leukemia is possible. As such, the invention provides a method and device for diagnosing tumor types. Nucleic acid probes or antibodies directed to various tumor markers are used as bioactive agents for the detection of tumor markers.

In an additional preferred embodiment the device finds use in transplantation analyses. The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body. The recognition of different foreign antigens is mediated by variable, antigen-specific regions of the T-cell receptor-analogous to the antibody reaction. In a graft rejection, the T-cells expressing a specific T-cell receptor which fits to the foreign antigen, could therefore be eliminated from the T-cell pool. Such analyses are possible by the identification of antigen-specific variable DNA sequences which are amplified by PCR and hence selectively increased. The specific amplification reaction permits the single cell-specific identification of a specific T-cell receptor. Similar analyses are presently performed for the identification of auto-immune disease like juvenile diabetes, arteriosclerosis, multiple sclerosis, rheumatoid arthritis, or encephalomyelitis.

In an additional preferred embodiment the device finds use in genome diagnostics. Four percent of all newborns are born with genetic defects; of the 3,500 hereditary diseases described which are caused by the modification of only a single gene, the primary molecular defects are only known for about 400 of them. Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations. Using direct and indirect DNA analysis, the diagnosis of a series of genetic diseases has become possible: sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, Huntington's chorea.

In an additional preferred embodiment the device finds use in pharmacogenomics. Pharmacogenomics has evolved from the academic science into an important tool for drug research and development. Accordingly, a new paradigm has evolved to target drug to patients with a specific genetic profile that predicts a favorable response to therapy. Different genes expression level of specific SNP's into certain genes can be useful for the treatment of cancer, diabetes and cardiovascular disease. Those candidate genes can be used to profile patients and their disease to allow for optimal treatment based on the presence or absence of specific genetic polymorphisms. By focusing on loci that appear to predict the onset of disease, it is the hope that pharmaceutical companies will intervene with new compounds designed to halt the progression of disease. When pharmacogenomics is integrated into drug research it allows pharmaceutical companies to stratify patient populations based on genetic background. During drug development, these same markers can be used to link efficacy or disease susceptibility to new pharmaceutical compounds. To be able to measure such changes in either single gene, many genes either as SNP or simple changes in expression level it requires a method as described to which may be utilized to overcome the challenges of modifying biological material such as DNA before measurement, enhance sample number throughput in a wide variety of based assays and overcome the used of highly specialized and expensive equipment.

In an additional preferred embodiment the device finds use in infectious disease. The application of recombinant DNA methods for diagnosis of infectious diseases has been most extensively explored for viral infections where current methods are cumbersome and results are delayed. In situ hybridization of tissues or cultured cells has made diagnosis of acute and chronic herpes infection possible. Fresh and fomalin-fixed tissues have been reported to be suitable for detection of papillomavirus in invasive cervical carcinoma and in the detection of HIV, while cultured cells have been used for the detection of cytomegalovirus and Epstein-Barr virus. The application of recombinant DNA methods to the diagnosis of microbial diseases has the potential to replace current microbial growth methods if cost-effectiveness, speed, and precision requirements can be met. Clinical situations where recombinant DNA procedures have begun to be applied include the identification of penicillin-resistant Neisseria gonorrhea by the presence of a transposon, the fastidiously growing chlamydia, microbes in foods; and simple means of following the spread of an infection through a population. The worldwide epidemiological challenge of diseases involving such parasites as leishmania and plasmodia is already being met by recombinant methods.

In an additional preferred embodiment the device finds use in gene expression analysis. One of the inventions disclosed herein is a high throughput method for measuring the expression of numerous genes (1-100) in a single measurement. The method also has the ability to be done in parallel with greater than one hundred samples per process. The method is applicable to drug screening, developmental biology, molecular medicine studies and the like. Thus, within one aspect of the invention methods are provided for analyzing the pattern of gene expression from a selected biological sample, comprising the steps of (a) exposing nucleic acids from a biological sample, (b) combining the exposed nucleic acids with one or more selected nucleic acid probes each located on a particular microsensor, under conditions and for a time sufficient for said probes to hybridize to said nucleic acids, wherein the hybridization correlative with a particular nucleic acid probe and detectable by the DNA-amplification-microsensor technology.

In additional preferred embodiments the device finds use in detection of micro-organisms, specific gene expression or specific sequences in nucleic acid. The use of DNA probes in combination with the DNA-amplification-microsensor technology can be used to detect the presence or absence of micro-organisms in any type of sample or specimen. Detectable nucleic acid can include mRNA, genomic DNA, plasmid DNA or RNA, rRNA viral DNA or RNA.

In an additional preferred embodiment the device finds use in mutation detection techniques. The detection of diseases is increasingly important in prevention and treatments. While multi factorial diseases are difficult to devise genetic tests for, more than 200 known human disorders are caused by a defect in a single gene, often a change of a single amino acid residue (Olsen, Biotechnology: An industry comes of age, National Academic Press, 1986). Many of these mutations result in an altered amino acid that causes a disease state.

Those point mutations are often called single-nucleotide polymorphisms (SNP) or cSNP when the point mutation are located in the coding region of a gene.

Sensitive mutation detection techniques offer extraordinary possibilities for mutation screening. For example, analyses may be performed even before the implantation of a fertilized egg (Holding and Monk, Lancet 3:532, 1989). Increasingly efficient genetic tests may also enable screening for oncogenic mutations in cells exfoliated from the respiratory tract or the bladder in connection with health checkups (Sidransky et al., Science 252:706, 1991). Also, when an unknown gene causes a genetic disease, methods to monitor DNA sequence variants are useful to study the inheritance of disease through genetic linkage analysis. However, detecting and diagnosing mutations in individual genes poses technological and economic challenges. Several different approaches have been pursued, but none are both efficient and inexpensive enough for truly widescale application.

Mutations involving a single nucleotide can be identified in a sample by physical, chemical, or enzymatic means. Generally, methods for mutation detection may be divided into scanning techniques, which are suitable to identify previously unknown mutations, and techniques designed to detect, distinguish, or quantitate known sequence variants, it is within that last described this invention has its strong advances compared to known status of the art technology.

Mutations are a single-base pair change in genomic DNA. Within the context of this invention, most such changes are readily detected by hybridization with oligonucleotides that are complementary to the sequence in question. In the system described here, two oligonucleotides are employed to detect a mutation. One oligonucleotide possesses the wild-type sequence and the other oligonucleotide possesses the mutant sequence. When the two oligonucleotides are used as probes on a wild-type target genomic sequence, the wild-type oligonucleotide will form a perfectly based paired structure and the mutant oligonucleotide sequence will form a duplex with a single base pair mismatch.

As discussed above, a 6 to 7° C. difference in the Tm of a wild type versus mismatched duplex permits the ready identification or discrimination of the two types of duplexes. To effect this discrimination, hybridization is performed at the Tm of the mismatched duplex in the respective hybotropic solution. The extent of hybridization is then measured for the set of oligonucleotide probes. When the ratio of the extent of hybridization of the wild-type probe to the mismatched probe is measured, a value to 10/1 to greater than 20/1 is obtained. These types of results permit the development of robust assays for mutation detection.

Other highly sensitive hybridization protocols may be used. The methods of the present invention enable one to readily assay for a nucleic acid containing a mutation suspected of being present in cells, samples, etc., i.e., a target nucleic acid. The “target nucleic acid” contains the nucleotide sequence of deoxyribonucleic acid (DNA) or ribonucleic acid (RNA) whose presence is of interest, and whose presence or absence is to be detected for in the hybridization assay. The hybridization methods of the present invention may also be applied to a complex biological mixture of nucleic acid (RNA and/or DNA). Such a complex biological mixture includes a wide range of eucaryotic and procaryotic cells, including protoplasts; and/or other biological materials which harbor polynucleotide nucleic acid. The method is thus applicable to tissue culture cells, animal cells, animal tissue, blood cells (e.g., reticulocytes, lymphocytes), plant cells, bacteria, yeasts, viruses, mycoplasmas, protozoa, fungi and the like. By detecting a specific hybridization between nucleic acid probes of a known source the specific presence of a target nucleic acid can be established.

An exemplary hybridization assay protocol for detecting a target nucleic acid in a complex population of nucleic acids is described as follows: A probe containing the SNP at the 3′ end is immobilized on one active SAW sensor at it's 5′ end (probe 1). Within the surroundings of the first micro-sensor a second SAW sensor is immobilized with a probe having the wild type sequence (probe 2). Two primer are designed for PCR amplification of a PCR product containing the potential SNP site. Normally the probe sites are located close to one of the primer sites. The following events may occur simultaneously in the chamber: 1) DNA amplification of target nucleic acid molecule in solution using the two above primers 2) hybridization of amplified target nucleic acid molecule to the probe 1 and probe 2 immobilized on two different SAW sensors. The target nucleic acid molecules are capable of hybridizing to the 3′ region of the immobilized probe sequence, to thereby form a hybridization complex that has a 3′ terminus; 3) 3′ extension of the DNA strand hybridized to the immobilized probe on the surface of the sensor to form a modified primer. If the DNA tested has the SNP site, probe 1 will hybridize more efficiently to the DNA compared to probe 2 where a 3′ mismatch will inhibit the 3′ extension reaction of the DNA strand hybridized to the immobilized probe on the surface of the cantilever. If the DNA tested does not contain SNP site (wild type), probe 2 will hybridize more efficiently to the DNA compared to probe 1 where a 3′ mismatch will inhibit the 3′ extension reaction of the DNA strand hybridized to the immobilized probe on the surface of the sensor. Those observations can be directly observed due to the frequency differences between the ouput signals of each SAW sensor, or the different frequency shifts between the input and output signal of each sensor.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### The Use of the SAWS-Immuno-Biosensor for Detection of Human Interleukin-6

Measurement Principle

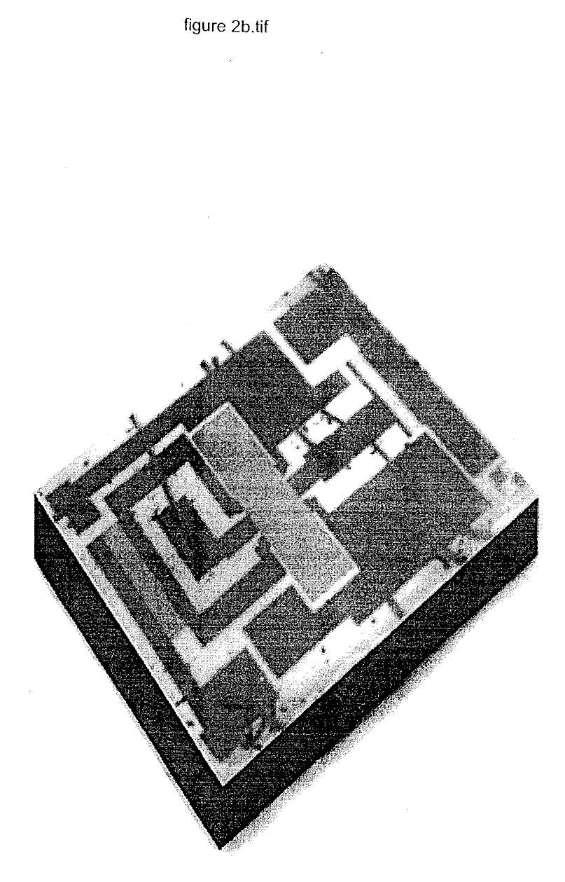

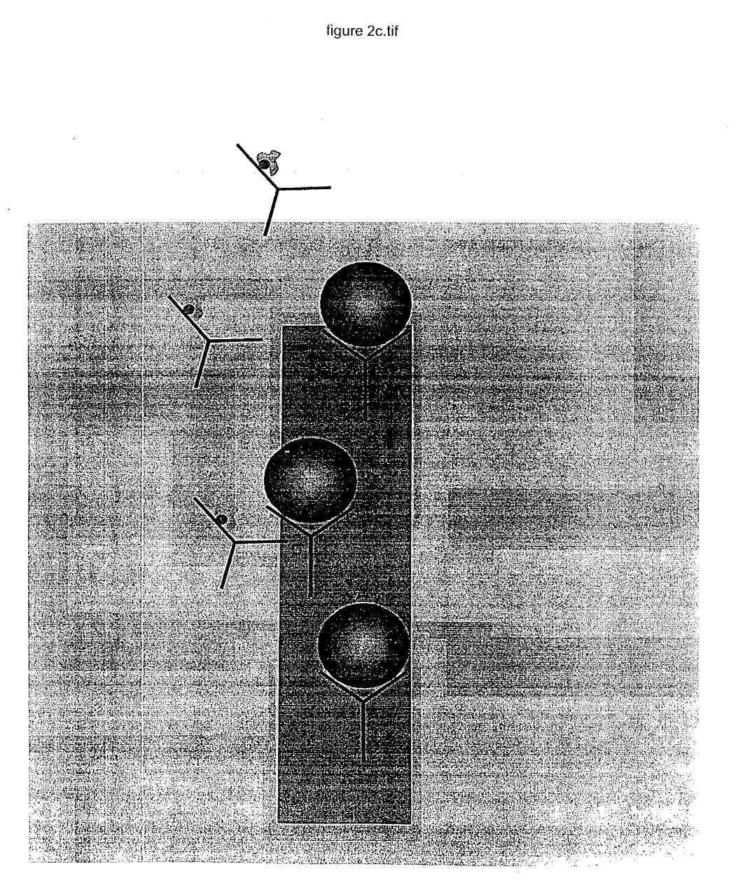

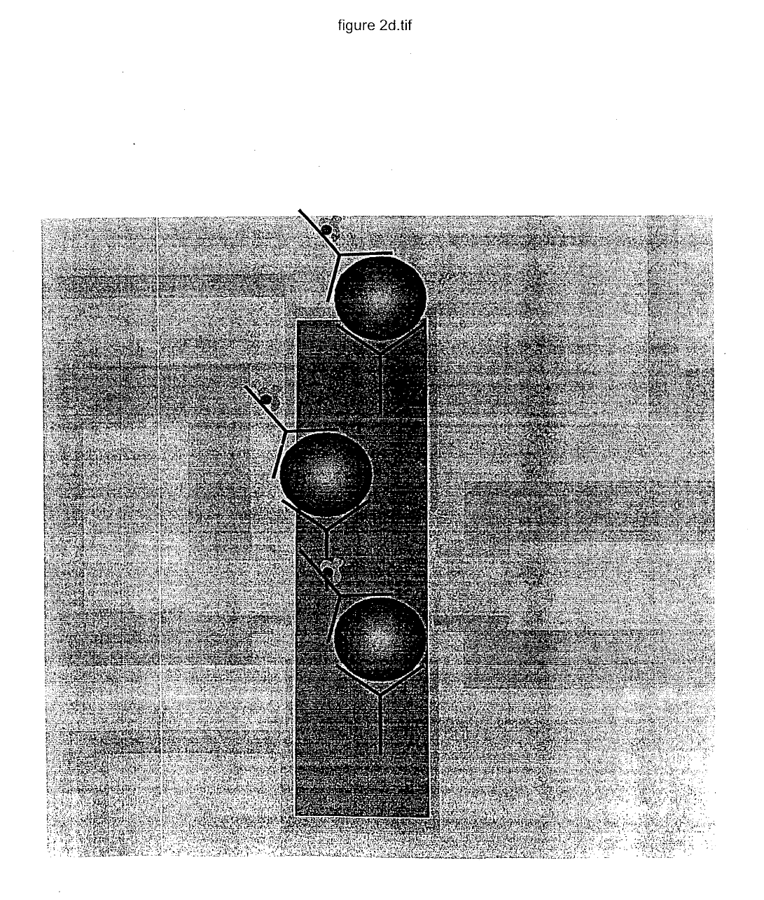

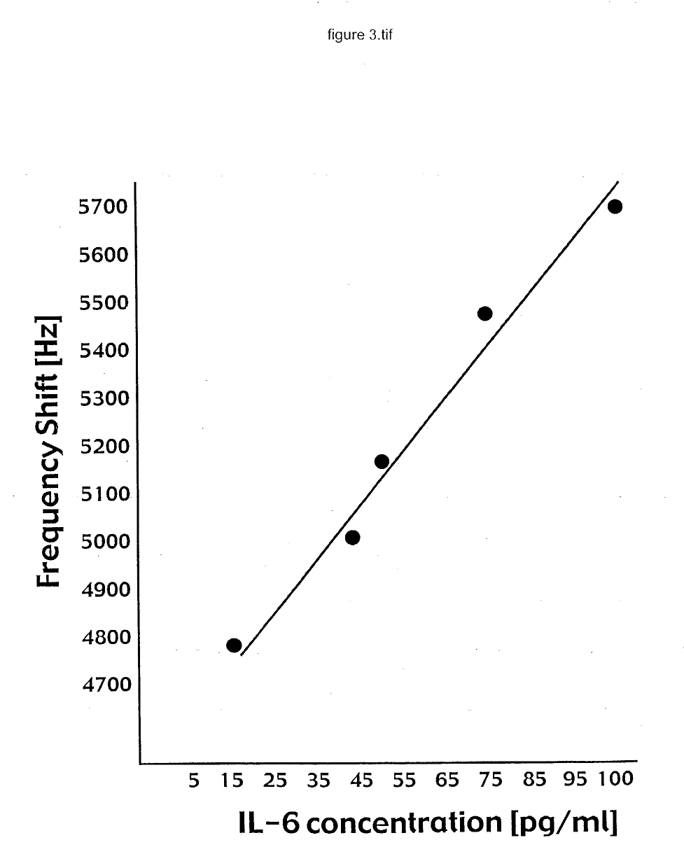

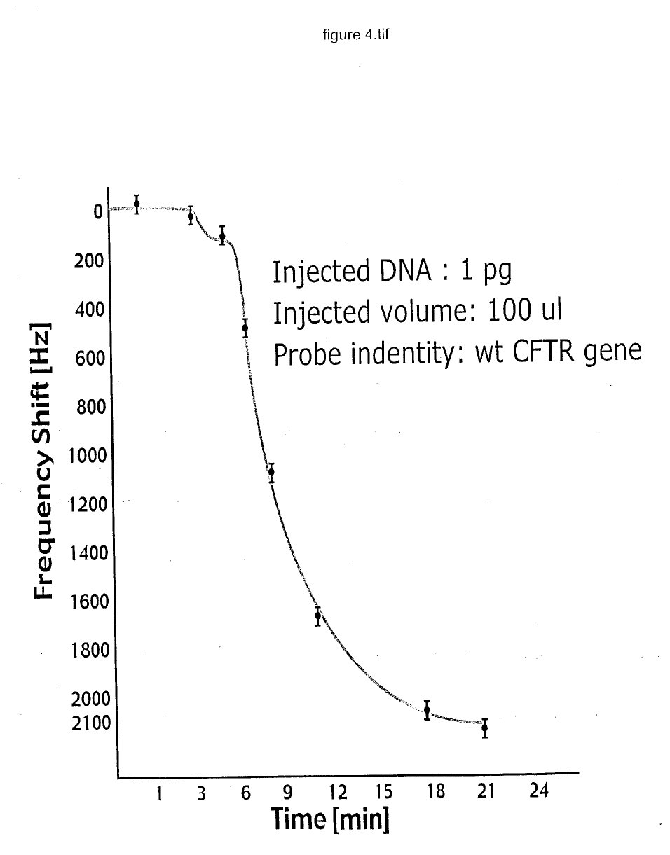

Anti-human IL-6 antibody is covalently assembled on a sensing surface between two interdigital transducers as illustrated in FIG. 2. This step is referred to as the programming procedure.

After the immunoreaction of the sample mix a protein complex of IL-6 anti-human antibody, IL-6 molecules, IL-6 biotinylated anti-human IL-6 antibody and avidin-horseradish peroxidase conjugate, will form on the sensor surface as illustrated in FIG. 2. When the protein complex is formed on the sensor surface, the acoustic wave will be delayed relative to the receiver interdigital transducers (IDTs) with a reduction of frequency as the end result. The Biosensor operates at 100 MHz in this example. To compensate for random event, each detector unit has two sets of sensing surfaces and two sets of interdigital transducers. One sensing surface is used for measurement of the specific hybridization event and the other sensing surface is only coated with a protein-blocking reagent, i.e., a reference sensor. Using an electronic differential amplifier, the difference between the two sensing surfaces is measured.

Production of SAWS-IL-6-Biosensors

The coating procedure: The sensor surface was coated with a polymer-shielding layer (polymide) to protect the metal surface and to guarantee the chemical stability of the IDT electrodes for the aqueous media. The polymer coating layer also functions as a wave-guide to trap the acoustic energy close to the surface.

The programming procedure: A carboxymethylated dextran layer was covalently attached to the polymer coated surface. The dextran layer served as a three dimensional universal matrix for attaching molecules containing NH₂ groups. The dextran layer was used for attaching the NH₂ groups of the anti-human IL-6 antibody to the surface of the SAWS-IL-6-Biosensor via activation of the carboxyl groups by N-hydroxysuccinimide and N-(3 dimethylaminopropyl)-N-ethylcarbodimide. Both the specific sensor surface and the reference sensor surface were blocked with 10% FBS according to the kit manual OptEIA™.

The assembling procedure: The first prototype SAWS-IL-6-Biosensor has incorporated a detector unit in a 100 μl microfluidic chip. The detector unit, which is programmed for detection of IL-6, is mounted at the bottom of the microfluidic chip. The liquid delivery system consist of an injection inlet, where 100 μl sample mix was injected into the sensing chamber.

Test Hardware, Experimental Setup

For initial SAWS-IL-6-Biosensor characterization, a network analyzer (HP 8751A) was used. A signal generator (HP8656B) and a vector voltmeter (HP8508A) were used for the sensing experiment, together with a switch control unit (HP3488A) and a multimeter (HP3457A). Alternatively, the different components may be built into one electronic unit consisting of both the driver Hz circuits and the sensor circuits.

Sample and Reagent Preparation

The antibodies used to measure IL-6 was obtained firn a commercially available ELISA kit (cat 555220/BD Bioscience). Using human recombinant IL-6 protein, serial dilutions were performed to gain concentration of the human IL-6 protein from 100 pg/ml to 15 pg/ml.

The sample mix consists of: IL-6 molecules (100 pg/ml to 15 pg/ml), biotinylated anti-human IL-6 antibody and avidin-horseradish peroxidase conjugate. The sample mix was injected into the SAWS-SNP-Biosensor. All measurement was performed at room temperature.

Results and Discussion

To test the sensitivity of the SAWS-IL-6-Biosensor, different recombinant IL-6 concentrations were used together with a detector unit having Anti-human IL-6 antibody covalently assembled on the sensing surface.

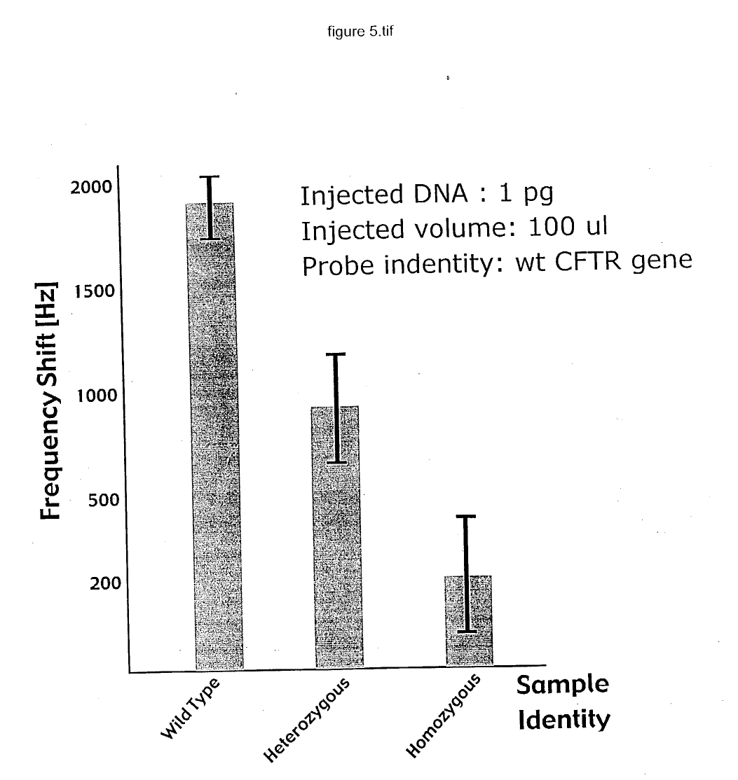

This application demonstrates that the SAWS-IL-6-Biosensor could be used for sensitivity immuno biosensor implementation in a liquid environment. It was shown that, in terms of sensitivity, 1.5 pg IL-6 gave (100 pg IL-6/ml) a frequency change of 4750 Hz, which gives a mass sensitivity as high as 0.3 fg/Hs as illustrated in FIG. 3. This sensitivity is comparable to the well established ELISA technology.

### The Use of the SAWS-Immuno-Biosensor for Genotyping of the Δ807 Mutation of the Cystic Fibrosis Gene

Measurement Principle

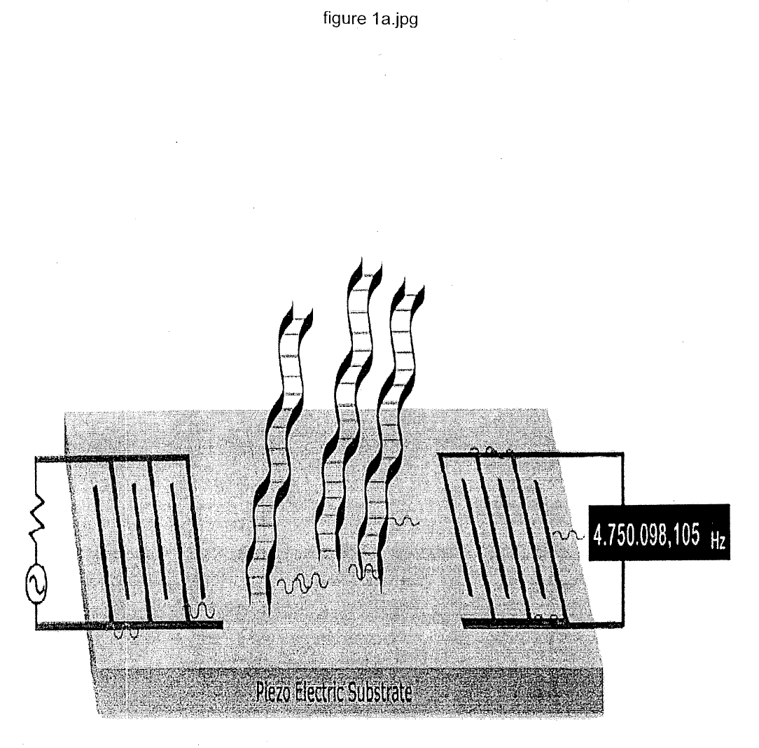

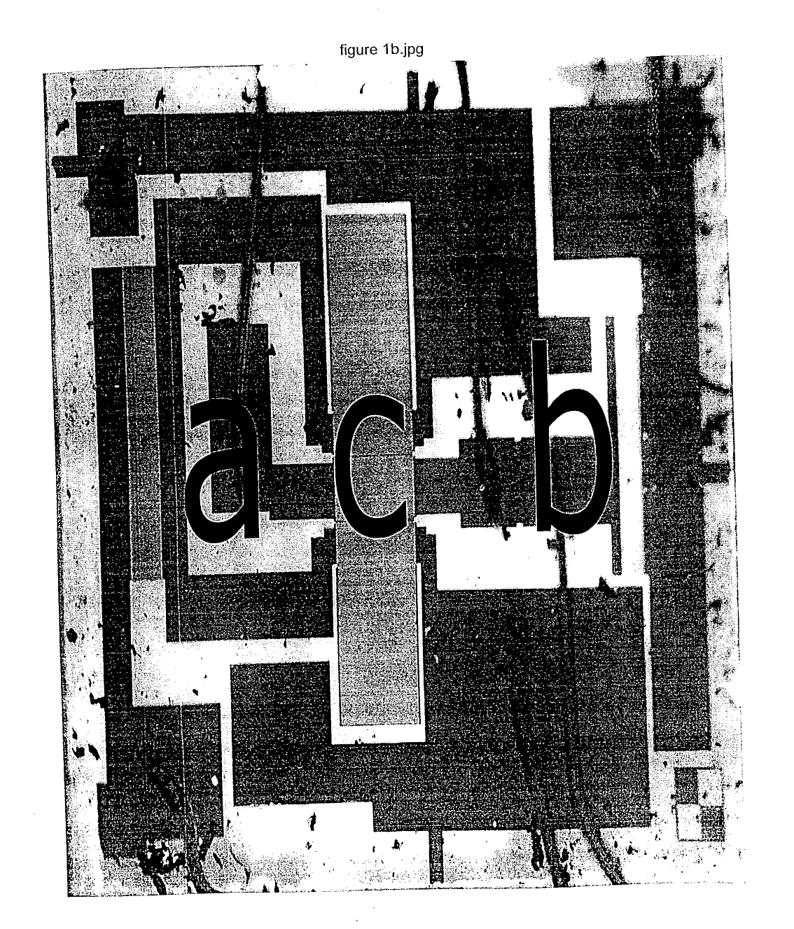

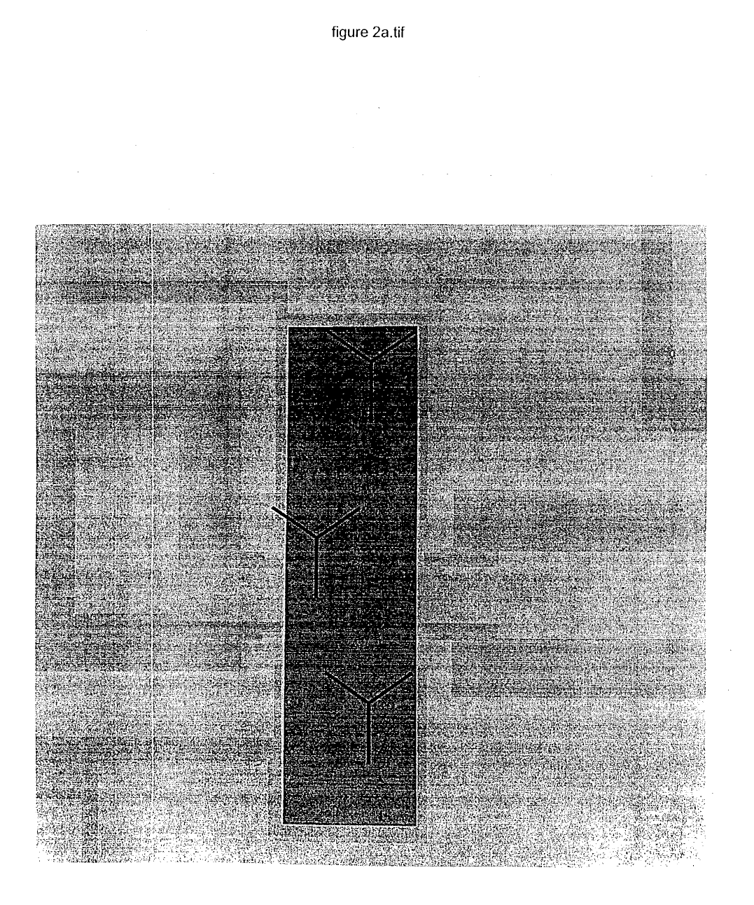

A NH₂ modified probe complementary to the target DNA is assembled on the sensing surface between two interdigital transducers as illustrated in FIG. 1. This step is referred to as the programming procedure.

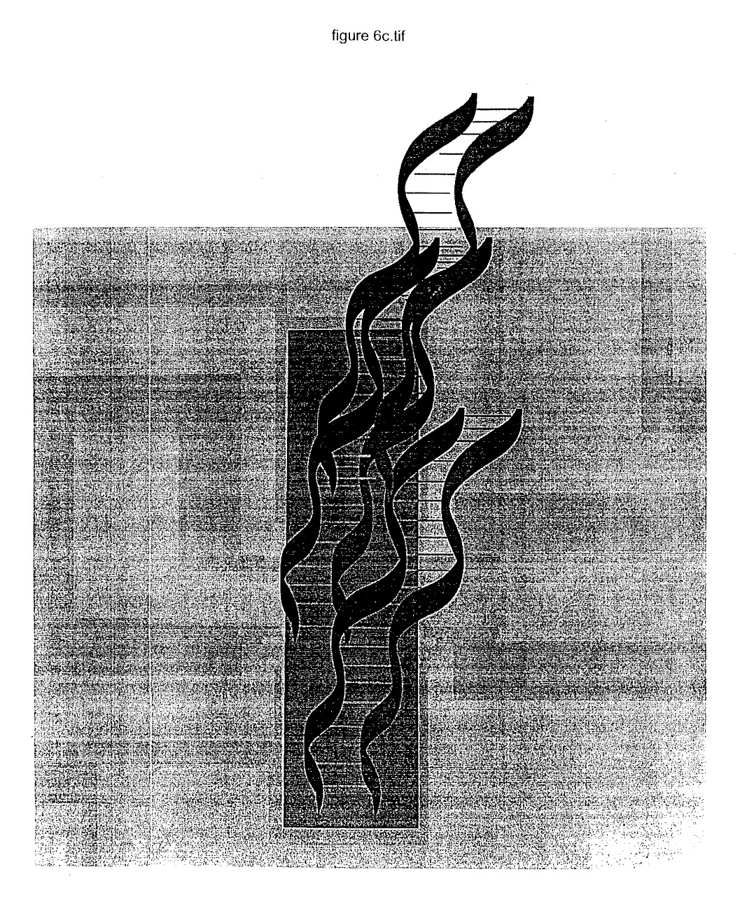

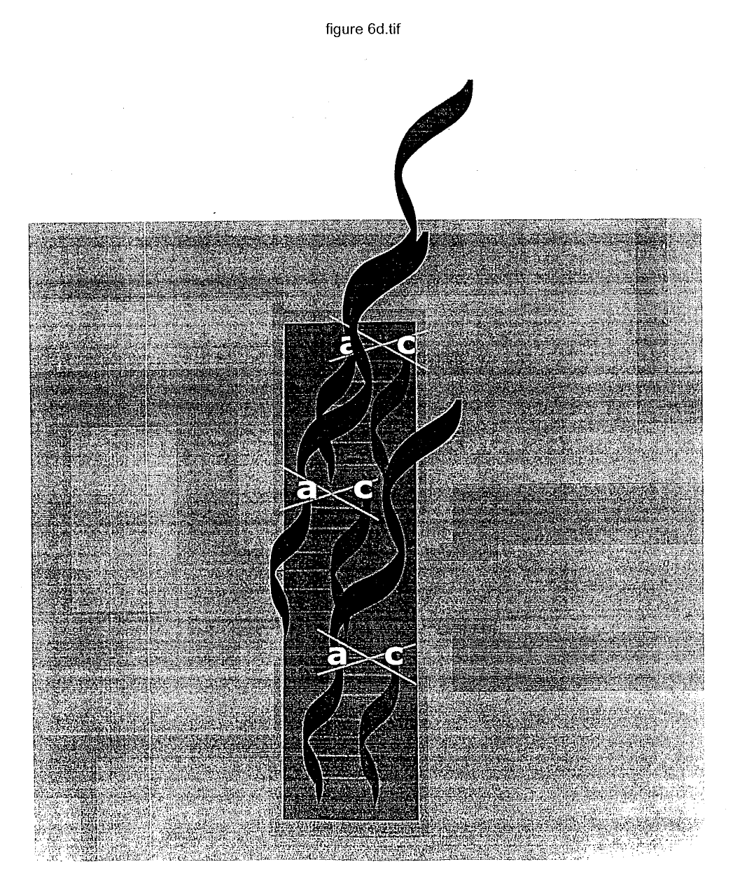

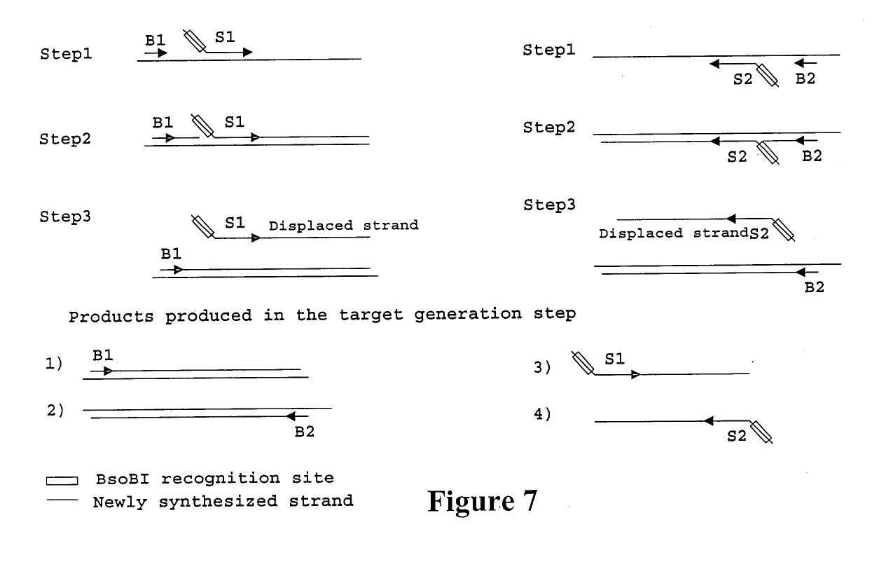

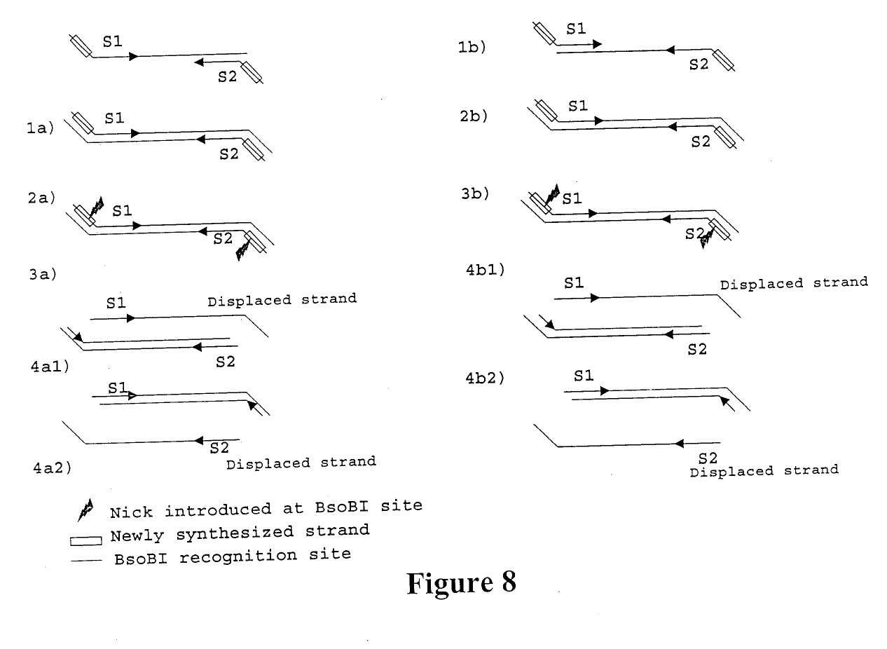

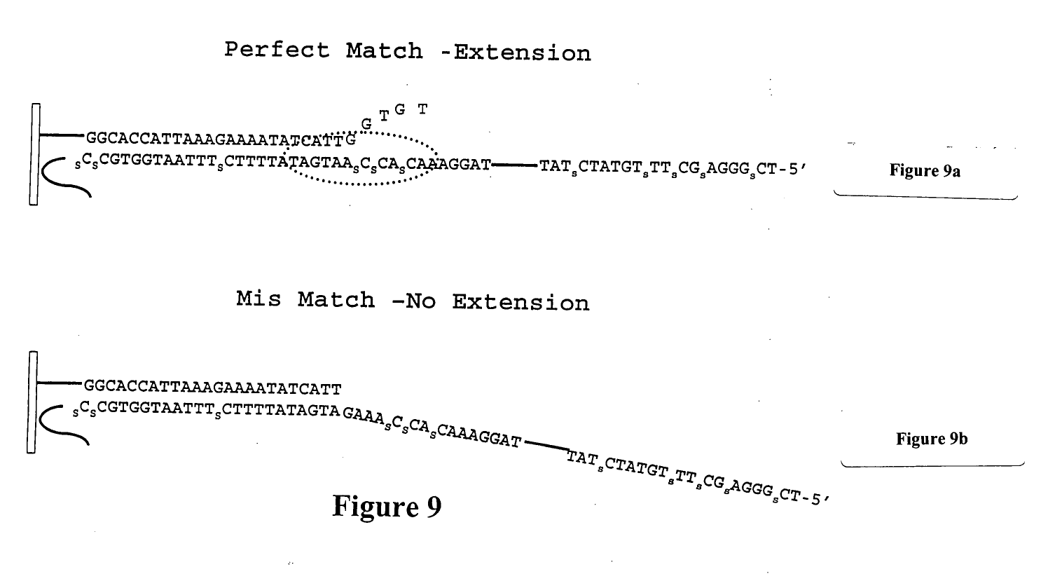

After hybridization between the probe and the target DNA, the DNA complex will be extended in the presence of a DNA polymerase. The DNA extension reaction will only take place if a perfect match exists between the 3′ end of the attached probe and the gene fragment as illustrated in FIG. 6. When the DNA extension reaction occurs on the sensor surface, the acoustic wave will be delayed relative to the receiver interdigital transducers (IDTs), with a reduction of frequency as the end result. The SAWS-SNP-Biosensor is operating at 100 MHz. To compensate for random event, each detector unit has two sets of sensing surfaces and two sets of interdigital transducers. One sensing surface is used for measurement of the specific hybridization event and the other sensing surface is probed with a random probe. Using an electronic differential amplifier, the difference between the two sensing surfaces is measured.

Production of SAWS-SNP-Biosensors

The Programming Procedure:

A carboxymethylated dextran layer was covalently attached to the polymer coated surface. The dextran layer served as a three dimensional universal matrix for attaching molecules containing NH2 groups. A gene specific NH2-modified oligonucleotide was attached to the dextran molecules via activation of the carboxyl groups by N-hydroxysuccinimide and N-(3 dimethylaminopropyl)-N-ethylcarbodiimide.

Blood Sample Preparation for A508-Mutation Detection

In preparation for the CFTR gene detection, Genomic DNA was purified from polymorphic blood samples using standard methods. A 599 bp DNA fragment from the C.FTR was amplified under standard PCR conditions. The 599 bp DNA fragment was visually inspected and the DNA concentration was measured OD 260/280. One pg DNA was incubated with lambda exonuclease for 15 minutes at 37° C. to obtain single stranded DNA. The volume was adjusted to 100 μl after adding DNA polymerase, dNTPs, biotin-11-dUTP and avidin. The DNA Mix was directly injected into the SAW-SNP-Biosensor unit.

Results and Discussion

In liquid phase applications, stability of the sensor platform is essential for reusability and reproducibility. Because the SAWS-SNP-Biosensor properties can be affected by the wave guide layer stability in water, different thicknesses of the coating layer have been tested. For the polymide polymer used, it was found that a thickness of 1800 nm has the optimal sensitivity (frequency shift) for DNA applications. Different tests were performed in terms of stability of the polymer attachment to the sensor surface. It was found that a sensor could be stored in liquid for several months without losing the protective polymer coating.

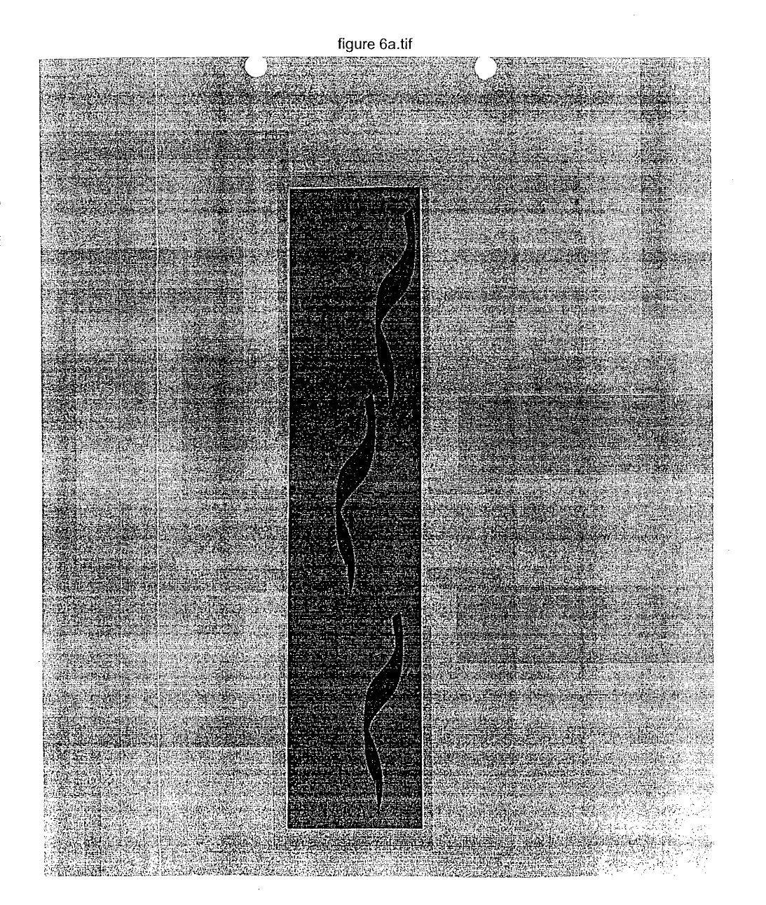

To test the sensitivity of the SAW-SNP-Biosensor, different DNA concentrations of wild type (wt) DNA were used together with a detector unit having a probe attached with a perfect wt match. As shown in FIG. 4, injection of 100 μl DNA Mix (1 pg DNA) with a concentration of 10 pg/ml gave after 10 minutes a frequency shift of 2.1 kHz. It can be observed from FIG. 4 that the curve has two shapes, which could be a mixture of two kinetics, where the first part of the curve represents the DNA extension event and the second part of the curve represents the DNA extension event.

Our results suggest that PCR amplification of the target DNA in the blood might not be necessary for measuring target DNA.

To test for selectivity, tree disposable SAWS-SNP-Biosensor units were used. The detector unit was programmed with one specific thiolated CFTR oligonucleotide, having a perfect match to the wt CFTR gene.

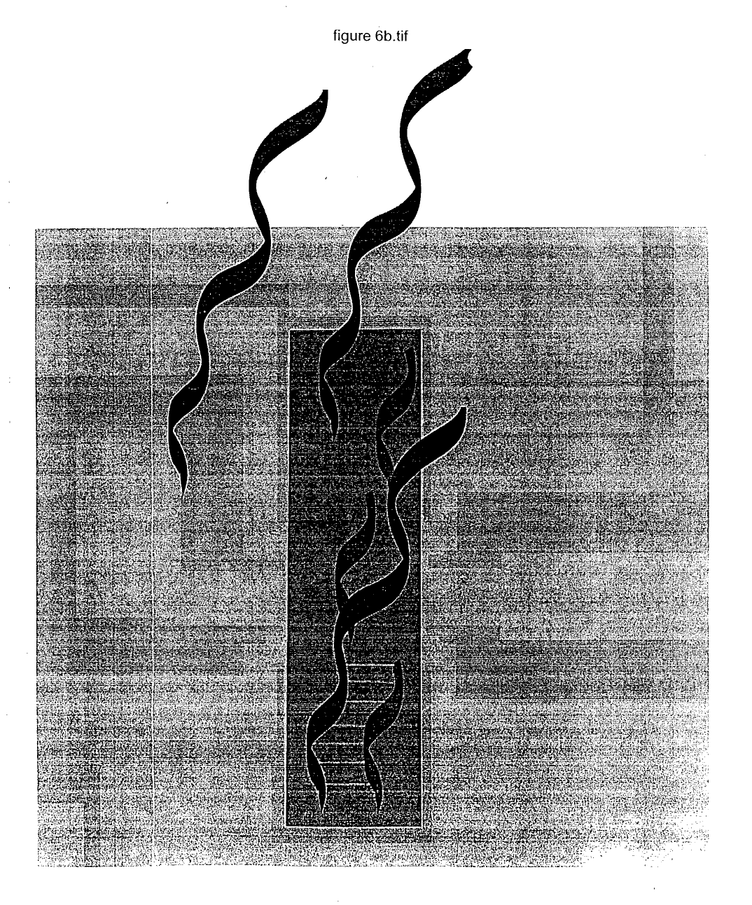

After injection of 1 pg DNA Mix from blood samples that were wt, heterozygous, or homozygous, we could clearly distinguish between the three polymorphic blood samples as illustrated in FIG. 5a, b and c.

This application demonstrates that the SAWS-SNP-Biosensor could be used for sensitivity sensor implementation in a liquid environment. It was shown that, in terms os sensitivity, 1 pg DNA gave a frequency change of 2100 Hz, which gives a mass sensitivity as high as 0.5 fg/Hz. This sensitivity is in the range where no amplification is needed to detect a particular gene using 50 μl full blood having approx. 7.5×106 white blood cells/ml full blood.

We recently have initiated a comparison between commonly used SNP detection methods and the SAWS-SNP-Biosensor technology to obtain clinical material to further investigate the sensitivity and selectivity of the SAWS-SNP-Biosensor.

The advantages compared to well known reference systems such as the LightCycler technology from Hoffmann La Roche seem to be: 1) label free technology, no need for chemical modification of clinical material; 2) ultimately we expect no need for amplification; 3) the SAWS-SNP-Biosensor can be stored at room temperature for a long period of time; 4) better standardization of the electronic measurement unit compared to flourescent and color detection instruments; 5) costs; cost per biosensor is expected to be very low.

### Strand Displacement Amplification

Description of Strand Displacement Amplification (SDA) technique;

Strand Displacement Amplification (SDA) is an isothermal method of nucleic acid amplification in which extension of primers, nicking of a hemimodified restriction endonuclease recognition/cleavage site, displacement of single stranded extension products, annealing of primers to the extension products (or the original target sequence) and subsequent extension of the primers occurs concurrently in the reaction mix. A bumper primer or external primer (B1 and B2) is a primer used to displace primer extension products in isothermal amplification reactions. The bumper primer anneals to a target sequence upstream of the amplification primer (S1 and S2) such that extension of the bumper primer displaces the downstream amplification primer and its extension product.

SDA is based upon 1) the ability of a restriction endonuclease to nick the unmodified strand of a hemiphosphorothioate form of its double stranded recognition/cleavage site and 2) the ability of certain polymerases to initiate replication at the nick and displace the downstream non-template strand. After an initial incubation at increased temperature (about 95.degree. C.) to denature double stranded target sequences for annealing of the primers, subsequent polymerization and displacement of newly synthesized strands takes place at a constant temperature. Production of each new copy of the target sequence consists of five steps: 1) binding of amplification primers to an original target sequence or a displaced single-stranded extension product previously polymerized, 2) extension of the primers by a 5′-3′ exonuclease deficient polymerase incorporating an alpha.-thio deoxynucleoside triphosphate (.alpha.-thio dNTP), 3) nicking of a hemimodified double stranded restriction site, 4) dissociation of the restriction enzyme from the nick site, and 5) extension from the 3′ end of the nick by the 5′-3′ exonuclease deficient polymerase with displacement of the downstream newly synthesized strand. Nicking, polymerization and displacement occur concurrently and continuously at a constant temperature because extension from the nick regenerates another nickable restriction site. When a pair of amplification primers is used, each of which hybridizes to one of the two strands of a double stranded target sequence, amplification is exponential. This is because the sense and antisense strands serve as templates for the opposite primer in subsequent rounds of amplification. When a single amplification primer is used, amplification is linear because only one strand serves as a template for primer extension.

The recognition site is for a thermophilic restriction endonuclease, BsoBI, so that the amplification reaction may be performed, with Bst DNA polymerase under conditions of thermoplilic SDA (tSDA).

1) The Target Generation Step.

Copies of double-stranded target sequence will be generated. The amplified products generated with Bumper primer, B1 and B2 will not contribute to further amplification (FIG. 1. Products 1 and 2), because of lack of nickable sites. However, the amplified product 3, generated with the amplification primer S1 and S2 are flanked with nickable BsoBI restriction sites (See also FIG. 1).

| | |
|---|---|
| BsoBI recognition site: | —C^{▾}TCGGG— |
| | —GAGCC_{▴}C— |

If Cytidine 5′-[alpha] Thiotriphosphate (CTPαS), is incorporated into the sequence, BsoBI will only make a nick between the C and T. and thereby create access for Bst DNA polymerase to synthesize a new template strand and displace one strand.

| | |
|---|---|
| Nickable BsoBI site: | —C^{▾}TₛCGGG— |
| | —GAGₛCₛCₛC— |

2) Amplification Step.

The product generated with S1 and S2 will be nicked at the flanked BsoBI restriction sites and thereby create access for Bst DNA polymerase, which will make access for the DNA polymerase to displace one strand while the syntheses of a new complementary strand is in process. (FIG. 2)

Amplification and detection of A508 or SNP's in Cystic Fibrosis.

The detector probe for A508 or SNP's in Cystic fibrosis is immobilized to the SAW surface. The detector CFTR probe 1 is lacking the codon-CTT-for Phenylalanine, which can course Cystic fibrosis. The oligonucleotide sequence for detector CFTR probe 2 is the DNA sequence for normal CFTR-gene.

The coding DNA sequence of Exon 10 of Human-CFTR gene; DNA Sequence in normal text: intron flanking exon 10.

DNA Sequence in italics: Exon 10 of CFTR

Amino-acid is shown in black with single letter abbreviations.

The codon-CTT-(A508) is shown in bold italics.

The placement of Bumper primers, B1 and B2 and the target binding site for

Amplification primers S1 and S2 are underlined.

```
TGACCTAATAATGATGGGTTTTATTTCCAGACTTCACTTCTAATGGTGATTATGGGAGAA -
                                                            T S L L M V I M G E
CTGGAGCCTTCAGAGGGTAAAATTAAGCACAGTGGAAGAATTTCATTCTGTTCTCAGTTT -
                                    B1 S1
L E P S E G K I K H S G R I S F C S Q F
TCCTGGATTATGCCTGGCACCATTAAAGAAAATATCATCTTTGGTGTTTCCTATGATGAA -
S W I M P G T I K E N I I F G V S Y D E
TATAGATACAGAAGCGTCATCAAAGCATGCCAACTAGAAGAGGTAAGAAACTATGTGAAA -
  S2 B2
Y R Y R S V I K A C Q L E E
```

The DNA Sequence of Primers (Table 1):

BsoBI recognition site is in bold. Target binding site is underlined.

| Upstream Bumper primer; | |
|---|---|
| B1 CFTR: | 5′-GGG TAA AAT TAA GCA GAG |
| Upstream Amplification primer; | |
| S1 CFTR: | 5′-ACC GCA TCG AAT GCA TGT CTC GGG ATC TCA GTT TTC CTG GA |
| Downstream Bumper primer | |
| B2 CFTR: | 5′-GTT TCT TAC CTC TTC TAG |
| Downstream Amplification primer; | |
| S2 CFTR: | 5′-CGA TTC CGC TCC AGA CTT CTC GGG AGA CGC TTC TGT ATC TAT |
| Immobilized detector probe; | |
| CFTR-Probe1: | 5′-HS-(CH₂)6 -CC CGA ATT CGG CAC CAT TAA AGA AAA TAT CAT T |
| CFTR-Probe2: | 5′-HS-(CH₂)6 -CC CGA ATT CGG CAC CAT TAA AGA AAA TAT CAT CCT |

In one method the CFTR detector probe 1 is immobilized to the Surface Acoustic Wave (SAW) biosensor surface. The target sequence is amplified by SDA in the solution. The complementary amplified target sequences, generated with the S2 Amplification primer, hybridize to the immobilized CFTR detector probe 1 at the SAW surface (FIG. 9). If a perfect match is present (D508), Bst DNA polymerase will extend the 3′ end of the immobilized CFTR detector probe 1 and a double-stranded DNA will be generated at the SAW surface (FIG. 9a). If a mismatch (normal gene) at the 3′-end is present, no extension will occur and the single-stranded DNA, generated with S2 amplification primer will remain single-stranded (FIG. 9b).

In another example the amplification primer S2 is internal labeled with an Amine group at an internal Thymidine.

BsoBI recognition site is in bold. Target binding site is underlined.

Downstream Amplification primer;
S2 CFTR:
5′-CGA TTC CGC TCC AGA CTT CTC GGG AGA CGC TTC
TGT^{NH2} ATC TAT

Gold particle are linked to the Thymidine with an Amine group. The target sequence is amplified by SDA, using the S2 Amplification primer labeled with gold particle and the amplification primer S1 (table 1) and Bumper primer B1 and B2 (table 1). The complementary amplified target sequence, generated with the S2 Amplification primer linked with a gold particle, hybridize to the immobilized CFTR detector probe 1 at the SAW surface (FIG. 10). If a perfect match (D508) is present, Bst DNA polymerase will extend the 3′ end of the immobilized CFTR detector probe 1 and a double-stranded DNA will be generated at the SAW surface (FIG. 10a). If a mismatch (normal gene) at the 3′-end is present, no extension will occur and the single-stranded DNA, generated with S2 amplification primer will remain single-stranded (FIG. 10b).

In another example the CFTR detector probe 2 is immobilized to the Surface Acoustic Wave (SAW) biosensor surface. The target sequence is amplified by SDA in the solution. The complementary amplified target sequences, generated with the S2 Amplification primer, hybridize to the immobilized CFTR detector probe 2 at the SAW surface (FIG. 11). If a perfect match (normal gene) is present, Bst DNA polymerase will extend the 3′ end of the immobilized CFTR detector Probe 2 and a double-stranded DNA will be generated at the SAW surface (FIG. 11a). If a mismatch (D508) at the 3′-end is present, no extension will occur and the single-stranded DNA, generated with S2 amplification primer will remain single-stranded (FIG. 11b).

In another example the amplification primer S2 is internal labeled with an Amine group at an internal Thymidine.

BsoBI recognition site is in bold. Target binding site is underlined.

Downstream Amplification primer;
S2 CFTR:
5′-CGA TTC CGC TCC AGA CTT CTC GGG AGA CGC TTC
TGT^{NH2} ATC TAT

Gold particle are linked to the Thymidine with an Amine group. The target sequence is amplified by SDA, using the S2 Amplification primer labeled with gold particle and the amplification primer S1 (table 1) and Bumper primer B1 and B2 (table 1). The complementary amplified target sequence, generated with the S2 Amplification primer linked with a gold particle, hybridize to the immobilized CFTR detector probe 2 at the SAW surface (FIG. 12). If a perfect match (normal gene) is present, Bst DNA polymerase will extend the 3′ end of the immobilized CFTR detector probe 1 and a double-stranded DNA will be generated at the SAW surface (FIG. 12a). If a mismatch (D508) at the 3′-end is present, no extension will occur and the single-stranded DNA, generated with S2 amplification primer will remain single-stranded (FIG. 12b).

What is claimed is:

1. A method for determining the presence or absence of a target nucleic acid in a test sample comprising:
contacting a target nucleic acid comprising first and second adjacent regions with a surface acoustic wave sensor comprising a surface having an immobilized probe nucleic acid which hybridizes to said first region of said test nucleic acid to form a hybridization complex, wherein said first region of said target nucleic acid is double stranded and said adjacent second region of said target nucleic acid is single stranded in said hybridization complex;
extending the probe nucleic acid in said hybridization complex using said second region in said test nucleic acid as template;
applying an input signal to an input transducer to generate a surface acoustic wave within said surface acoustic wave sensor;
receiving said surface acoustic wave at an output transducer;
generating an electronic output signal; and
measuring a parameter of said output signal which provides an indication of whether or not said target nucleic acid is present in said test sample.

2. The method of claim 1, wherein said parameter is chosen from the group consisting of frequency and phase of said output signal.

3. The method of claim 1, further comprising:
providing a reference surface acoustic wave sensor comprising a surface having a reference surface, wherein said reference surface does not specifically bind said target nucleic acid;
applying an input signal to a second input transducer to generate a reference surface acoustic wave within said reference surface acoustic wave sensor;
receiving said reference surface acoustic wave at a reference output transducer, generating a reference output signal;
measuring a parameter of said reference output signal and detecting the difference between said parameter of said output signal and said reference output signal.

4. The method of claim 3, wherein said parameter is chosen from the group consisting of frequency shift and phase shift of said reference output signal.

5. The method of claim 1 wherein extension of said probe nucleic acid is dependent upon base pair matches or mismatches with one or more opposing nucleotides in said first or said second region.

6. The method of claim 5 wherein said base pair match or mismatch is not the terminal nucleotide position of said probe nucleic acid.

7. The method of claim 1 further comprising amplifying said target nucleic acid.

8. The method of claim 7 wherein said amplifying is by RCA, PCR or LCR and said amplification occurs simultaneously with said contacting.

9. The method of claim 7 wherein the presence of a base pair match or mismatch at said terminal nucleotide in said hybridization complex is indicative of the substitution, insertion or deletion of one or more nucleotides in said test nucleic acid as compared to said probe nucleic acid.

10. The method of claim 1 wherein the measurement of said parameter provides an indication of the concentration of said target nucleic acid in said test sample.
